(19) United States
(12) Patent Application Publication
(10) Pub. No.: US 2009/0143332 A1
(43) Pub. Date: Jun. 4, 2009

(54) Cyclodextrin Cyanohydrins

(76) Inventors: Mikael Bols, Virum (DK); Fernando Ortega Caballero, Granada (ES)

Correspondence Address: HARNESS, DICKEY & PIERCE, P.L.C., P.O. BOX 8910, RESTON, VA, 20195, US

(21) Appl. No.: 11/883,997

(22) PCT Filed: Feb. 9, 2006
(86) PCT No.: PCT/DK06/00075

§ 371 Date: Jun. 6, 2008

(30) Foreign Application Priority Data
Feb. 10, 2005 (EP) 05075335.9

(51) Int. Cl. C08B 37/16; C07H 15/20; A61K 31/724; A61P 1/00; A61P 25/30

(52) U.S. Cl. 514/58; 536/46; 536/18.5

(57) ABSTRACT

The present invention relates to a compound having a cyclodextrin skeleton wherein a hydrogen atom at the C-6 position in at least one of the sugar moieties has been substituted with a cyano group thereby forming a cyanohydrin-type group. The compounds are found to be excellent catalysts, e.g. for the hydrolysis of aryl glycosides. Accordingly, the compounds of the invention are useful as medicaments, in particular for the treatment of poisoning and drug abuse. The compounds, optionally immobilized to a solid phase material, are useful to reduce the content of harmful substances, e.g. metabolites of fungi, insects, etc., from compositions such as foodstuff.

### FIELD OF THE INVENTION

The present invention relates to novel compounds, namely compounds of the cyclodextrin cyanohydrin-type, the use of such compounds as medicaments, e.g., for treating conditions caused by poisoning or drug abuse, and to the use of the compounds as catalysts.

### BACKGROUND OF THE INVENTION

It is well-known to use small molecules as therapeutics. Virtually all known therapeutics act as passive ligands being, e.g., enzyme inhibitors or receptor agonists/antagonists. This limits the action of these compounds to the interference with the chemical machinery of the human organism. An entirely new idea is to use small molecules that can act as enzymes as active therapeutics that render harmful substances, such as toxins or addictive drugs, harmless. There are many toxic glycosides and other harmful substances that can be rendered harmless by hydrolysis. A relatively small molecule that can catalyse this hydrolysis would fulfil this requirement.

Cyclodextrins are small carbohydrate molecules of bacterial origin that can complex other molecules and are known to be able to act as enzyme-like catalysts (Breslow, R.; Dong, S. D. Chem. Rev. 1998, 98, 1997-2011). They consist of 6-8 glucose molecules joined together in a ring by α-1,4-glycosidic linkanges (see FIG. 1). α-cyclodextrin consists of 6 glucose units, β-cyclodextrin consists of 7 glucose units, and γ-cyclodextrin consists of 8 glucose units. It has been shown that cyclodextrins and cyclodextrin derivatives can catalyse the hydrolysis of glycosides. Ohe et al. (Ohe, T.; Kajiwara, Y.; Kida, T.; Zhang, W.; Nakatsuji, Y.; Ikeda, I. Chem. Lett. 1999, 921-922) observed that α-cyclodextrin increased the rate of hydrolysis of 4-nitrophenyl α-mannopyranoside at pH 12 up to 7.6 times, while the hydrolysis of the β-anomer was unaffected. Conversion of other nitrophenyl glycosides was increased up to 8.6 fold. β-cyclodextrin did not affect the hydrolysis. Doug et al. (Doug, T. H.; Chou, J. Z.; Huang, X.; Bennet, A. J. J. Chem. Soc., Perkin Trans. 2 2001, 83-89.) investigated the hydrolysis of 4-nitrophenyl 2-tetrahydropyranyl ether, a model of a glycoside, catalysed by α- and β-cyclodextrin. They found that α-cyclodextrin accelerated the hydrolysis about 4 fold, while β-cyclodextrin decreased the rate of hydrolysis. A β-cyclodextrin derivative with a single 2-O-carboxymethyl group likewise decreased the hydrolysis rate. They also observed that β-cyclodextrin catalysed the hydrolysis of a 2-deoxyglucopyranosyl pyridinium salt with k_{cat}/k_{uncat}≧7.5. In these examples, the catalytic effect is low and the conditions are quite extreme compared to the physiological conditions in the mammalian body.

### BRIEF DESCRIPTION OF THE INVENTION

It has surprisingly been discovered that compounds of the cyclodextrin cyanohydrin-type are remarkable good catalysts of glycoside hydrolysis at neutral pH increasing the hydrolysis rate (k_{cat}/k_{uncat}) with up to more than 2000, which is a significant improvement compared to the hydrolysis rates reported in the prior art. The invention, thus, relates to compounds of the cyclodextrin cyanohydrin-type, which are novel compounds, and their various uses as therapeutics and catalysts.

More specifically, the invention relates to a compound having a cyclodextrin skeleton wherein a hydrogen atom at the C-6 position in at least one of the sugar moieties has been substituted with a cyano group.

The present invention also relates to a pharmaceutical composition comprising a compound as defined herein, and a pharmaceutically acceptable carrier, excipient or diluent therefor.

The present invention further relates to a compound as defined herein for use as a medicament, and more particularly to the use of such a compound for the preparation of a medicament for the treatment of poisoning, or for the treatment of drug addiction.

Accordingly, the invention also relates to a method of treating a mammal suffering from poisoning, or alternatively suffering from drug addiction, wherein said method comprising the step of administering a compound as defined herein to said mammal.

Still further, the present invention also relates to the use of a compound as defined herein as a catalyst. Accordingly, the invention also relates to a method of hydrolysing an aryl glycoside, wherein said hydrolysis is carried out in the presence of such a compound.

Also, the present invention relates to a solid phase material having immobilized thereto a compound as defined herein.

Finally, the present invention also relates to a method of reducing or eliminating the content of aryl glycosides in a composition, said method comprising the steps of contacting the composition with a compound as defined herein, or a solid phase material having immobilized thereto such a compound, under conditions suitable for effecting hydrolysis of said aryl glycosides.

### BRIEF DESCRIPTION OF THE DRAWINGS

FIG. 1 illustrates the chemical structure of typical cyclodextrins and the numbering used in cyclodextrin and cyclodextrin derivatives.

FIG. 2 illustrates the reactions performed in Example 1.

FIG. 3 illustrates a Hanes plot S/V vs S showing that compound 7 catalyse the hydrolysis of 3 different nitrophenyl glycosides in an enzyme-like manner.

FIG. 4 illustrates progress curve for the hydrolysis of 4-nitrophenyl β-D-glucopyranoside (10 mM) at pH 7.4, 59° C. in presence of different concentration of 7 (0.01-0.1 mM).

FIG. 5 illustrates the proposed mechanism for the catalysis.

### DETAILED DESCRIPTION OF THE INVENTION

As mentioned above, the invention relates to a compound having a cyclodextrin skeleton wherein a hydrogen atom at the C-6 position in at least one of the sugar moieties has been substituted with a cyano group.

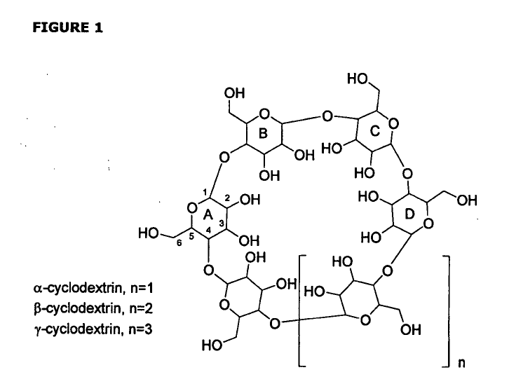

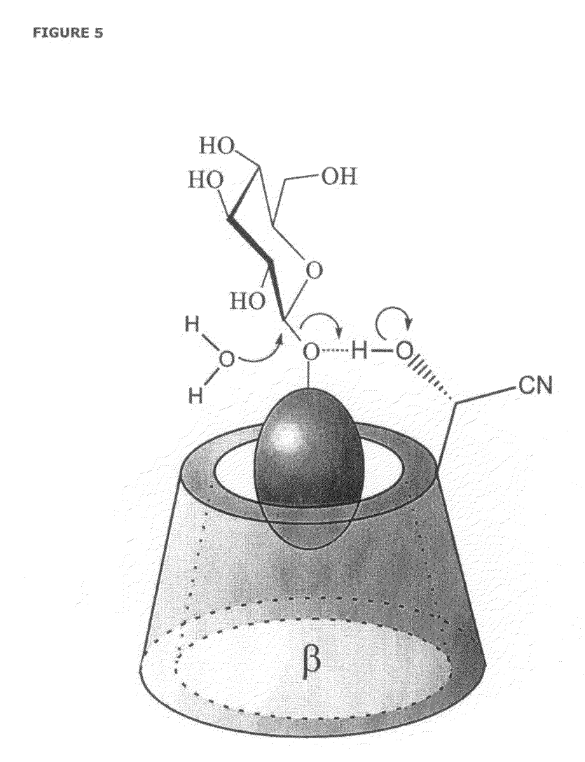

As mentioned hereinabove, cyclodextrin compounds are cyclic compounds. In the present context, the term “cyclodextrin skeleton” is intended to mean that the respective compound consists of 5-10 sugar moieties (preferably 6-8 sugar moieties), in particular glucose molecules, joined together in a ring by α-1,4-glycosidic linkages (see, e.g., FIG. 1), thereby forming a barrel-like structure (see FIG. 5). Examples hereof are α-cyclodextrin consisting of 6 glucose units, β-cyclodextrin consisting of 7 glucose units, and γ-cyclodextrin consisting of 8 glucose units. It should be understood that the sugar moieties (preferably glucose moieties) of the compounds defined herein may be modified in various ways as will be apparent from the following. This may include alteration of the stereochemistry.

A crucial feature of the compound of the invention is the presence of a cyanohydrin-type group in what corresponds to the C-6 position of one or more of the glucose moieties. It can be said that a hydrogen atom at the C-6 position has been substituted with a cyano group thereby rendering the C-6 position into a group of the type:

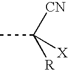

wherein X and R are as defined herein. It is believed that the substituent X preferably should represent a heteroatom substituent, thus each X may, e.g., independently be selected from hydroxy, amino, thio, mono- or di(C₁₋₈-alkyl)amino, C₃₋₈-cycloalkylamino, C₁₋₈-acylamino, and trifluoroacetylamino.

The cyano group possesses electron-withdrawing properties and renders any hydrogen atom of the group X more acidic. The R group may be hydrogen or may be a group that further modifies the acidity of the hydrogen atom of the X group. Thus, the R groups are preferably, if not a hydrogen atom, an electron-withdrawing substituent, or alternatively a hydroxy group.

In some of the preferred embodiments, each of the X groups are independently selected from substituents having an acidic hydrogen attached to the heteroatom, e.g. like in hydroxy, thio, amino and mono(C₁₋₈-alkyl)amino. In such instances, R is preferably hydrogen.

### EMBODIMENTS

In view of the above and the results described in the “Experimentals” section, it has been found that the compounds of the invention preferably have the general structure I

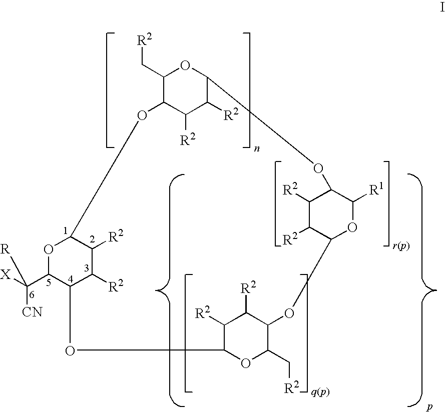

wherein
n is an integer of 0-9,
each q(p) is an integer of 0-2,
each r(p) is an integer of 1-2, and
p is an integer of 0-5,
with the proviso that the sum (1+n+Σ{q(x)+r(x)}_{=1, . . . , p}) is 5-10, in particular 6-8;

each R independently is selected from hydrogen, cyano, hydroxy, C₁₋₈-alkyl, C₃₋₈-cycloalkyl, C₂₋₈-alkenyl, CF₃, C₁₋₈-alkylcarbonyloxy, carboxy, and mono- or di(C₁₋₈-alkyl)aminocarbonyl;

each X independently is selected from heteroatom substituents;

each R¹ independently is selected from optionally substituted C₁₋₈-alkyl, optionally substituted C₃₋₈-cycloalkyl, optionally substituted C₂₋₈-alkenyl, mono- or di-(C₁₋₄-alkyl)amino, tri(C₁₋₈-alkyl)ammonium, carboxy, carboxaldehyde, optionally substituted aryl, and a group

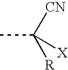

where R and X are defined as above (in particular CH(OH)CN);

each R² independently is selected from hydrogen, hydroxy, optionally substituted C₁₋₈-alkoxy, optionally substituted aryloxy, optionally substituted arylmethyloxy, optionally substituted C₁₋₈-acyloxy, tri-substituted silyloxy, O-phosphate and O-sulphate, or two R² substituents on neighbouring carbon atoms form an O,O-acetal group;

and salts thereof.

The integers n, p, q(p) and r(p) define the total number of sugar moieties in the ring by means of the sum (1+n+Σ{q(x)+r(x)}_{x=1, . . . , p}) being 5-10, in particular 6-8. The integers also define the position of the sugar moieties having the C-6 cyanohydrin-type group. The general structure II is encompassed by the general structure I, e.g where n=n; p=m+1; q(1), . . . , q(m)=1; q(p)=0; r(p)=1.

Although the stereochemical configuration of the substituents R¹ and R² is not defined in the general structures I and II (see further below), it is preferred that the substituents R¹ and R² in each of the sugar moieties represent the D-gluco, manno, altro or allo configuration, in particular the D-gluco configuration, where the indicated configurations relate to the 6-membered ring.

Each R is independently selected from hydrogen, cyano, hydroxy, C₁₋₈-alkyl, C₃₋₈-cycloalkyl, C₂₋₈-alkenyl, CF₃, C₁₋₈-alkylcarbonyloxy, carboxy, and mono- or di(C₁₋₈-alkyl)aminocarbonyl. In many instances, each R is hydrogen.

Each X is independently selected from heteroatom substituents. The term “heteroatom substituent” means that the atom immediately adjacent to the C-6 is a heteroatom, e.g. oxygen, sulfur, nitrogen, etc. Examples of heteroatom substituents are those selected from hydroxy, amino, thio, mono- or di(C₁₋₈-alkyl)amino, C₃₋₈-cycloalkylamino, C₁₋₈-acylamino, and trifluoroacetylamino. Preferably, the substituent carries a hydrogen atom, i.e. preferred substituents are hydroxy (—OH), thio (—SH), amino (—NH₂), mono(C₁₋₈-alkyl)amino (—NH(C₁₋₈-alkyl)), and C₃₋₈-cycloalkylamino. In combination with a cyano group, the latter substituents resembles the functionality of a cyanohydrin.

Each R¹ is independently selected from optionally substituted C₁₋₈-alkyl, optionally substituted C₃₋₈-cycloalkyl, optionally substituted C₂₋₈-alkenyl, mono- or di-(C₁₋₄-alkyl)amino, tri(C₁₋₈-alkyl)ammonium, carboxy, carboxaldehyde, optionally substituted aryl, and a group

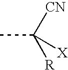

where R and X are defined as above.

Preferably, each R¹ is a group

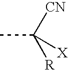

wherein X is a heteroatom carrying a hydrogen atom, thereby defining further C-6 groups having the cyanohydrin functionality.

In some preferred embodiments, the compounds of the invention comprise 2-4, in particular 2, cyanohydrin functionalities, i.e. 2-4 groups of the type

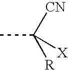

wherein X is selected from hydroxy (—OH), thio (—SH), amino (—NH₂), mono(C₁₋₈-alkyl)amino (—NH(C₁₋₈-alkyl)) and C₃₋₈-cycloalkylamino.

In other preferred embodiments, the compounds of the invention comprise 1 cyanohydrin functionality, i.e. 1 group of the type

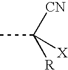

wherein X is selected from hydroxy (—OH), thio (—SH), amino (—NH₂), mono(C₁₋₈-alkyl)amino (—NH(C₁₋₈-alkyl)) and C₃₋₈-cycloalkylamino. Within these embodiments, it is preferred that the R¹ groups present independently are selected from optionally substituted C₁₋₈-alkyl, optionally substituted C₃₋₈-cycloalkyl, optionally substituted C₂₋₈-alkenyl, mono- or di-(C₁₋₄-alkyl)amino, tri(C₁₋₈-alkyl)ammonium, carboxy, carboxaldehyde, and optionally substituted aryl.

Each R² is independently selected from hydrogen, hydroxy, optionally substituted C₁₋₈-alkoxy, optionally substituted aryloxy, optionally substituted arylmethyloxy, optionally substituted C₁₋₈-acyloxy, tri-substituted silyloxy, O-phosphate and O-sulphate, or two R² substituents on neighbouring carbon atoms form an O,O-acetal group (e.g. by condensation of the corresponding hydroxy (2×R²) compound with an aldehyde (e.g. benzaldehyde, acetaldehyde, etc.) or a ketone (e.g. acetone). The selection of the R² groups does not appear to be particularly critical. In many instances, it is preferred that each R² is hydroxy. Otherwise, such a hydroxy group may be blocked (e.g. by an optionally substituted C₁₋₈-alkoxy or optionally substituted aryloxy) or protected (optionally substituted arylmethyloxy, optionally substituted C₁₋₈-acyloxy, tri-substituted silyloxy, O-phosphate and O-sulphate, or two R² substituents on neighbouring carbon atoms form an O,O-acetal group). Such protection groups may not necessarily be removed before use of the compound for its intended use, although this is normally preferred due to solubility considerations.

In one preferred embodiment, the compound is one wherein each X independently is selected from hydroxy, amino, thio, C₁₋₈-alkylamino and C₃₋₈-cycloalkylamino; and each R¹ independently is a group

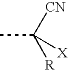

where R and X are defined as above, in particular a group selected from CH(OH)CN, CH(SH)CN and CH(NH₂)CN, in particular CH(OH)CN.

In a more specific embodiment, the compound is one which has the general structure II

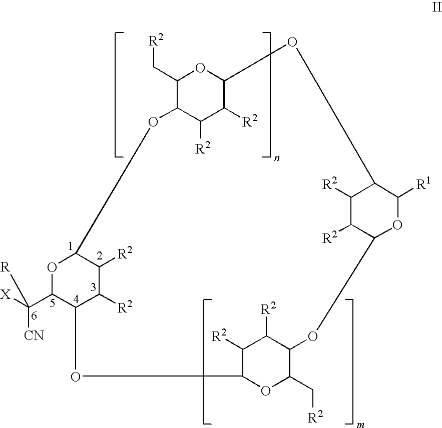

wherein n, R, X, R¹ and R² are as defined above, and m is an integer from 0 to 4, with the proviso that the sum (2+m+n) is 5-10, in particular 6-8.

Among the compounds of the general structures I and II, it is preferred that each R¹ independently is a group

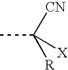

and R is hydrogen and X is selected from hydroxy, thio and amino, in particular hydroxy.

A particular subclass of compounds of the general structures I and II is the one wherein each X independently is selected from hydroxy, thio and amino; each R is hydrogen; and each R¹ independently is selected from CH(OH)CN, CH(SH)CN and CH(NH₂)CN, in particular CH(OH)CN.

Also preferred is the embodiment where each R² independently is selected from hydrogen, hydroxy, protected hydroxy and C₁₋₄-alkoxy, in particular hydroxy.

In a preferred version, the A and D ring of the cyclodextrin (see FIG. 1) each carry a cyano group.

Thus with reference to the most promising results, it is believed that particularly interesting compounds are those wherein each X is OH; each R independently is selected from hydrogen, C₁₋₈-alkyl and C₃₋₈-cycloalkyl (in particular hydrogen); each R¹ independently is selected from C₁₋₈-alkyl optionally substituted with hydroxy or C₁₋₄-alkoxy, carboxy, carboxaldehyde, and the group

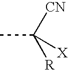

where R and X are defined as above, in particular CH(OH)CN; and each R² independently is selected from hydroxy and C₁₋₄-methoxy; in particular those of the general structure II wherein X is OH; R is hydrogen; R¹ is CH(OH)CN; R² is hydroxy; and m is 2 and n is 2 or 3, namely structure III

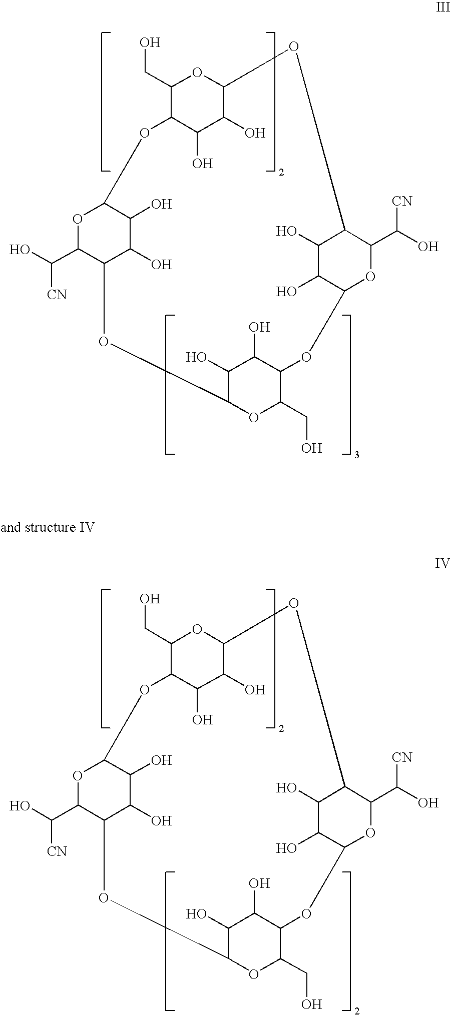

### DEFINITIONS

In the present context, the term “C₁₋₈-alkyl” is intended to mean a linear or branched hydrocarbon group having 1 to 8 carbon atoms, such as methyl, ethyl, propyl, iso-propyl, pentyl, hexyl, heptyl and octyl, and the term “C₁₋₄-alkyl” is intended to cover linear or branched hydrocarbon groups having 1 to 4 carbon atoms, e.g. methyl, ethyl, propyl, iso-propyl, butyl, iso-butyl, and tert-butyl.

The term “alkoxy” means “alkyl-oxy”, i.e. “alkyl-O—”.

The term “C₃₋₈-cycloalkyl” is intended to mean a cyclic hydrocarbon group having 3 to 8 carbon atoms, such as cyclopropyl, cyclobutyl, cyclopentyl, cyclohexyl, etc.

Similarly, the term “C₂₋₈-alkenyl” is intended to cover linear or branched hydrocarbon groups having 2 to 6 carbon atoms and comprising one unsaturated bond. Examples of alkenyl groups are vinyl, allyl, butenyl, pentenyl, hexenyl, heptenyl and octenyl. Preferred examples of alkenyl are vinyl, allyl, butenyl, especially allyl.

In the present context, i.e. in connection with the terms “alkyl”, “cycloalkyl”, “alkoxy”, and “alkenyl”, the term “optionally substituted” is intended to mean that the group in question may be substituted one or several times, preferably 1-3 times, with group(s) selected from hydroxy (which when bound to an unsaturated carbon atom may be present in the tautomeric keto form), C₁₋₆-alkoxy (i.e. C₁₋₆-alkyl-oxy), C₂₋₆-alkenyloxy, carboxy, oxo (forming a keto or aldehyde functionality), C₁₋₆-alkylcarbonyl, formyl, aryl, aryloxy, arylamino, arylcarbonyl, amino, mono- and di(C₁₋₆-alkyl)amino; carbamoyl, mono- and di(C₁₋₆-alkyl)aminocarbonyl, amino-C₁₋₆-alkyl-aminocarbonyl, mono- and di(C₁₋₆-alkyl)amino-C₁₋₆-alkyl-aminocarbonyl, C₁₋₆-alkylcarbonylamino, guanidino, carbamido, C₁₋₆-alkyl-sulphonyl-amino, C₁₋₆-alkyl-sulphonyl, C₁₋₆-alkyl-sulphinyl, C₁₋₆-alkylthio, halogen, where any aryl may be substituted as specifically described below. In some embodiments, substituents are selected from hydroxy, C₁₋₆-alkoxy, amino, mono- and di(C₁₋₆-alkyl)amino, carboxy, C₁₋₆-alkylcarbonylamino, C₁₋₆-alkylaminocarbonyl, or halogen.

The term “halogen” includes fluoro, chloro, bromo, and iodo.

In the present context, the term “aryl” is intended to mean a fully or partially aromatic carbocyclic ring or ring system, such as phenyl, naphthyl, 1,2,3,4-tetrahydronaphthyl, anthracyl, phenanthracyl, pyrenyl, benzopyrenyl, fluorenyl and xanthenyl, among which phenyl is a preferred example.

In the present context, i.e. in connection with the term “aryl” and the like (e.g. “aryloxy”), the term “optionally substituted” is intended to mean that the group in question may be substituted one or several times, preferably 1-5 times, in particular 1-3 times, with group(s) selected from hydroxy, C₁₋₆-alkyl, C₁₋₆-alkoxy, oxo (which may be represented in the tautomeric enol form), carboxy, C₁₋₆-alkylcarbonyl, formyl, amino, mono- and di(C₁₋₆-alkyl)amino; carbamoyl, mono- and di(C₁₋₆-alkyl)aminocarbonyl, amino-C₁₋₆-alkyl-aminocar-bonyl, C₁₋₆-alkylcarbonylamino, guanidino, carbamido, C₁₋₆-alkyl-sulphonyl-amino, aryl-sulphonyl-amino, C₁₋₆-alkyl-suphonyl, C₁₋₆-alkyl-sulphinyl, C₁₋₆-alkylsulphonyloxy, sulphanyl, amino, amino-sulfonyl, mono- and di(C₁₋₆-alkyl)amino-sulfonyl or halogen, where any alkyl, alkoxy and the like, representing substituents may be substituted with hydroxy, C₁₋₆-alkoxy, C₂₋₆-alkenyloxy, amino, mono- and di(C₁₋₆-alkyl)amino, carboxy, C₁₋₆-alkylcarbonylamino, halogen, C₁₋₆-alkylthio, C₁₋₆-alkyl-sulphonyl-amino, or guanidino. In some embodiments, the substituents are selected from C₁₋₆-alkyl, C₁₋₆-alkoxy, amino, mono- and di(C₁₋₆-alkyl)amino, sulphanyl, carboxy or halogen, where any alkyl, alkoxy and the like, representing substituents may be substituted with hydroxy, C₁₋₆-alkoxy, C₂₋₆-alkenyloxy, amino, mono- and di(C₁₋₆-alkyl)amino, carboxy, C₁₋₆-alkylcarbonylamino, halogen, C₁₋₆-alkylthio, C₁₋₆-alkyl-sulphonyl-amino, or guanidino.

The term “trisubstituted silyl” is intended to mean a group of the formula (Z¹)(Z²)(Z³)Si—, wherein each of Z¹, Z² and Z³ independently is selected from C₁₋₈-alkyl and aryl.

The term “salts” is intended to include acid addition salts and basic salts. Illustrative examples of acid addition salts are pharmaceutically acceptable salts formed with non-toxic acids. Exemplary of such organic salts are those with maleic, fumaric, benzoic, ascorbic, succinic, oxalic, bis-methylenesalicylic, methanesulfonic, ethanedisulfonic, acetic, propionic, tartaric, salicylic, citric, gluconic, lactic, malic, mandelic, cinnamic, citraconic, aspartic, stearic, palmitic, itaconic, glycolic, p-aminobenzoic, glutamic, benzenesulfonic, and theophylline acetic acids, as well as the 8-halotheophyllines, for example 8-bromotheophylline. Exemplary of such inorganic salts are those with hydrochloric, hydrobromic, sulfuric, sulfamic, phosphoric, and nitric acids. Examples of basic salts are salts where the (remaining) counter ion is selected from alkali metals, such as sodium and potassium, alkaline earth metals, such as calcium, and ammonium ions (⁺N(R′)₃R″, where R′ and R″ independently designates optionally substituted C₁₋₆-alkyl, optionally substituted C₂₋₆-alkenyl, optionally substituted aryl, or optionally substituted heteroaryl). Pharmaceutically acceptable salts are, e.g., those described in Remington's Pharmaceutical Sciences, 17. Ed. Alfonso R. Gennaro (Ed.), Mack Publishing Company, Easton, Pa., U.S.A., 1985 and more recent editions and in Encyclopedia of Pharmaceutical Technology. Thus, the term “an acid addition salt or a basic salt thereof” used herein is intended to comprise such salts. Furthermore, the compounds as well as any intermediates or starting materials may also be present in hydrate form.

### Preparation of the Compounds

The compounds of the invention can be prepared by methods known per se by the skilled art worker, for example as described in the following and in the Experimentals section. Starting from commercially available α-, β- or γ-cyclodextrin, benzylation (according to Sato, T.; Nakamra, H.; Ohno, Y.; Endo, T. Carbohydr. Res., 1990, 199, 31-35) gives the perbenzylated cyclodextrin that is selectively debenzylated in either one or two 6-positions (according to Pearce, A. J.; Sinaÿ, P. Angew. Chem. Int. Ed. 2000, 39, 3610-3612). The resulting alcohols are oxidised with Dess-Martins reagent, reacted with potassium cyanide and the benzyl groups are removed by hydrogenolysis (see example below) to give a compound of the invention.

Alternatively, the cyanohydrin can, before the final debenzylatlon, be reacted with mesyl chloride/pyridine converting it into an α-cyanomesylate that then is substituted with azide, halogen, amine, thiol or cyanide that then is debenzylated to a compound of the invention.

Alternatively, the aldehyde, obtained from Dess-Martin oxidation as described above, is reacted with an organometallic reagent giving another alcohol which then is oxidised again to give a ketone. The resulting ketone can then be processed as described above being reacted with potassium cyanide to give a cyanohydrin and debenzylation by hydrogenolysis yielding another compound of the invention.

Alternatively, the partially benzylated diol can be monosilylated with tert-butyldimethylsilyl chloride/imidazol. The resulting monool can now be oxidised to aldehyde or acid, substituted with halogen and further substituted with other nucleophiles such as cyanide, azide, thiol or amine. After desilylation the remaining monool can be processed as described above to give a cyanohydrin thereby giving a compound of the invention having one cyanohydrin and one other functional group in the 6-positions of the A and D residues.

After the above synthesis, a compound of the invention may be further modified by substitution or protection of one or more of the many hydroxyl groups to yield another compound of the invention. Many methods are known according to which a single, several or all hydroxyl groups of a cyclodextrin derivative may be protected with alkyl, silyl, sulfonyl or acyl groups (see Khan, A. R.; Forgo, P.; Stine, K. J.; D'Souza, Valerian T. Chem. Rev. 1998, 98, 1977-1996). Also one or more hydroxyl groups can be substituted with halogen. Further substitution of halogen or sulfonyl with hydrogen, amine, thiol or azide can give another compound of the invention.

### Catalysts

The compounds of the invention can be used as catalysts, e.g. for the production of compounds under mild conditions. Treatment of many substances, such as glycosides, esters, amides and other compounds that can be hydrolysed with aqueous acid, with the compound of invention and water will lead to hydrolysis of the substance under very mild, neutral condition allowing sensitive, valuable compounds to be produced. Alternatively, water can be substituted with a protic solvent or a nucleophile in this reaction allowing the compound of the invention to catalyse a trans-glycosidation, trans-esterification or similar.

Thus, one preferred aspect of the invention relates to a method of hydrolysing an aryl glycoside, wherein said hydrolysis is carried out in the presence of a compound as defined herein (optionally immobilized as described in the following).

It is believed that the compounds are generally useful for hydrolysis reactions, such as hydrolysis of glycosides, esters, and amides, as well as for trans-glycosidation and trans-esterification reactions.

When used as a catalyst, it is believed to be advantageous if the compound defined herein is immobilised to a solid phase material so that separation from the reaction mixture can be effected smoothly after completion of the catalysed reaction.

Thus, the present invention also relates to a solid phase material having immobilized thereto a compound as defined herein. It is envisaged that immobilization can be accomplished by covalently linking the compound, e.g. via the C-2, C-3 or C-6 position (e.g. via a C-2, C-3 or C-6 hydroxy group) of a glucose unit of the cyclodextrin. Suitable solid phase materials, generally applicable linker types and the general methods of immobilizing compounds to a solid phase material are thoroughly described in the literature and will be well-known to the person skilled in the art.

The catalytic action of the compound of the invention may also be utilized for other purposes than chemical synthesis, e.g. in connection with composition either comprising aryl glycosides or being contaminated with aryl glycosides. Thus, the invention also provides a method of reducing or eliminating the content of aryl glycosides in a composition, said method comprising the steps of contacting the composition with a compound as defined herein, or a solid phase material having immobilized thereto such a compound, under conditions suitable for effecting hydrolysis of said aryl glycosides. In one preferred embodiment, the composition is a foodstuff (e.g. dried fruit, drinking water, vegetables, grains, etc.).

### Various Medical Uses

The compounds of this invention can be used as therapeutics, in particular to treat acute toxicity. There are many toxic glycosides in plants (e.g. Laburnum sp., Solanum sp., etc.), insects, snakes, fungi and other organisms that are responsible for poisoning humans and livestock that eat them. Administration of the compounds of this invention to a poisoned individual or livestock is believed to facilitate hydrolysis of the toxic substance thereby rendering it harmless and neutralising the poison.

Alternatively, the compounds of this invention can be used as therapeutics to treat addiction or an overdose of an addictive substance such as cocaine. Administration of the compounds of the invention to an addict which has an addictive substance, such as cocaine, in the bloodstream will lead to hydrolysis of the substance rendering it unable to enter the brain and exert its physiological or toxic effects. Since the addict has no reward from the hallucinatory substance he will have no craving.

Thus, the present invention also relates to a compound as defined herein for use as a medicament.

Furthermore, the present invention relates to the use of a compound as defined herein for the preparation of a medicament for the treatment of poisoning, and to the use of a compound as defined herein for the preparation of a medicament for the treatment of drug addiction.

Similarly, the invention also provides a method of treating a mammal suffering from poisoning, said method comprising the step of administering a compound as defined herein to said mammal, and a method of treating a mammal suffering from drug addiction, said method comprising the step of administering a compound as defined herein to said mammal.

### Pharmaceutical Compositions

In a further aspect, the invention relates to a pharmaceutical composition comprising a compound as defined herein and pharmaceutically acceptable carrier, excipient or diluent therefor.

The compound is suitably formulated in a pharmaceutical composition so as to suit the desirable route of administration.

The administration route of the compounds may be any suitable route which leads to a concentration in the blood or tissue corresponding to a therapeutic effective concentration. Thus, e.g., the following administration routes may be applicable although the invention is not limited thereto: the oral route, the parenteral route, the cutaneous route, the nasal route, the rectal route, the vaginal route and the ocular route. It should be clear to a person skilled in the art that the administration route is dependent on the particular compound in question; particularly the choice of administration route depends on the physico-chemical properties of the compound together with the age and weight of the patient and on the particular disease or condition and the severity of the same.

The compounds may be contained in any appropriate amount in a pharmaceutical composition, and are generally contained in an amount of about 1-90%, e.g. 1-10%, by weight of the total weight of the composition. The composition may be presented in a dosage form which is suitable for the oral, parenteral, rectal, cutaneous, nasal, vaginal and/or ocular administration route. Thus, the composition may be in form of, e.g., tablets, capsules, pills, powders, granulates, suspensions, emulsions, solutions, gels including hydrogels, pastes, ointments, creams, plasters, drenches, delivery devices, suppositories, enemas, injectables, implants, sprays, aerosols and in other suitable form. Due to the fact that the compounds are particularly useful for acute conditions, a preferred route of administration is by injection.

The pharmaceutical compositions may be formulated according to conventional pharmaceutical practice, see, e.g., “Remington's Pharmaceutical Sciences” and “Encyclopedia of Pharmaceutical Technology”, edited by Swarbrick, J. & J. C. Boylan, Marcel Dekker, Inc., New York, 1988. Typically, the compounds defined herein are formulated with (at least) a pharmaceutically acceptable carrier, excipient or diluent. Pharmaceutically acceptable carriers, excipients, and diluents are those known by the person skilled in the art. Formation of suitable salts of the compounds of the Formula I will also be evident in view of the before-mentioned.

Preparation of solid dosage forms for oral use, controlled release oral dosage forms, fluid liquid compositions, parenteral compositions, controlled release parenteral compositions, rectal compositions, nasal compositions, percutaneous and topical compositions, controlled release percutaneous and topical compositions, and compositions for administration to the eye will be well-known to those skilled in the art of pharmaceutical formulation. Specific formulations can be found in “Remington's Pharmaceutical Sciences”.

Capsules, tablets and pills etc. may contain for example the following compounds: microcrystalline cellulose, gum or gelatine as binders; starch or lactose as excipients; stearates as lubricants; various sweetening or flavouring agents. For capsules the dosage unit may contain a liquid carrier such as fatty oils. Likewise, coatings of sugar or enteric agents may be part of the dosage unit. The pharmaceutical compositions may also be emulsions of the compound(s) and a lipid forming a micellular emulsion.

For parenteral, subcutaneous, intradermal or topical administration the pharmaceutical composition may include a sterile diluent, buffers, regulators of tonicity and antibacterials. The active compound may be prepared with carriers that protect against degradation or immediate elimination from the body, including implants or microcapsules with controlled release properties. For intravenous administration, the preferred carriers are physiological saline or phosphate buffered saline.

### Dosages

The dosage of the compound according to the invention depends on the compound in question; however, the amount of the compound is also closely related to the therapeutic agent co-administered with the compound as well as the dosage of said therapeutic agent.

For all methods of use disclosed herein for the compounds, the daily oral dosage regimen will preferably be from about 0.01 to about 80 mg/kg of total body weight. The daily parenteral dosage regimen will be from about 0.001 to about 80 mg/kg of total body weight.

The term “unit dosage form” as used herein refers to physically discrete units suitable as unitary dosages for human and animal individuals, each unit containing a predetermined quantity of a compound, alone or in combination with other agents, calculated in an amount sufficient to produce the desired effect in association with a pharmaceutically acceptable diluent, carrier, or vehicle. The specifications for the unit dosage forms of the present invention depend on the particular compound or compounds employed and the effect to be achieved, as well as the pharmacodynamics associated with each compound in the host. The dose administered should be an “effective amount” or an amount necessary to achieve an “effective level” in the individual patient.

Since the “effective level” is used as the preferred endpoint for dosing, the actual dose and schedule can vary, depending on inter-individual differences in pharmacokinetics, drug distribution, and metabolism. The “effective level” can be defined, for example, as the blood or tissue level desired in the individual that corresponds to a concentration of one or more compounds according to the invention. Also, the effective level is depending on the therapeutic agent in question, and in particular on the concentration of the effective level in question.

Accordingly, in a preferred embodiment the ratio of the compound administered to the therapeutic agent administered is in the interval of from 200:1 mol:mol to 1:200 mol:mol, such as in the interval of from 100:1 mol:mol to 1:50 mol:mol, such as in the interval of from 50:1 mol:mol to 1:25 mol:mol

The compound may be administered in any suitable dosage regime, but is preferably administered with the same intervals as the therapeutic agent, preferably either shortly before or during administration of the therapeutic agent.

Most of the therapeutic agents according to this invention are administered parenterally, often intravenously. The compound according to the invention may be administered in any suitable manner according to the formulation thereof, it is however often preferred that the compound is administered parenterally, such as intravenously as the therapeutic agent.

### EXPERIMENTALS

### Materials and Method

All reagents were used as purchased without further purification. TLC was performed on Merck Silica Gel 60 F₂₅₄ plates with detection by charring with cerium sulphate and ammonium heptamolybdat, and by UV light when applicable. Flash column chromatography was performed on Silica Gel Fluka (230-400 mesh) as stationary phase. Optical rotations were recorded on a Perkin-Elmer 241 polarimeter at room temperature. IR spectra were recorded on a Perkin-Elmer FT-IR PARAGON 1000. ¹H and ¹³C NMR spectra were recorded on a Varian Mercury 400 MHz spectrometer. Chemical shifts are given in ppm and referenced to internal SiMe₄ (δ_{H}, δ_{C} 0.00). J values are given in Hz. MALDI-TOF Mass spectra were recorded on a Voyager DE PRO mass spectrometer (Applied Biosystems) using an α-cyanohydroxycinnamic acid (α-CHCA) matrix. Spectra were calibrated with angiotensin I m/z 1296.69, adrenocorticotropic hormone (ACTH) (clip 1-17) m/z 2093.09, ACTH (clip 18-39) m/z 2465.20, and ACTH (clip 7-38) m/z 3657.93.

### EXAMPLE 1

### Preparation of Cyclodextrin Cyanohydrins

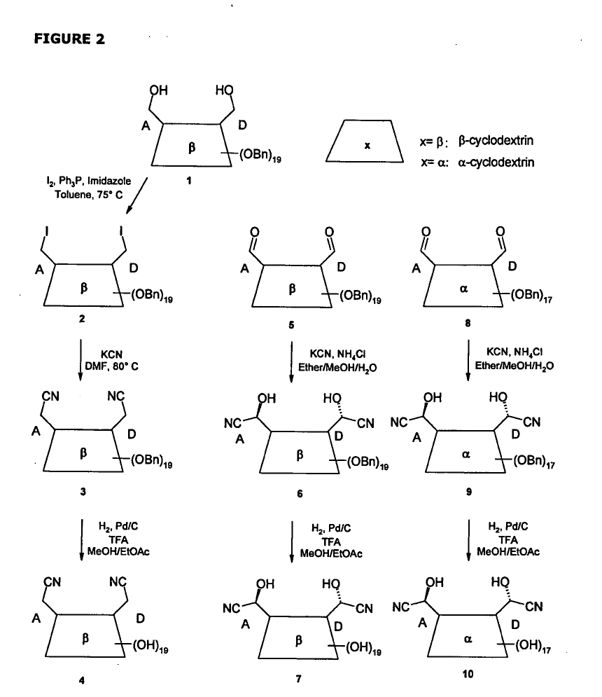

The synthesis is outlined in FIG. 2.

### A. Preparation of Disubstituted Cyclodextrins

6^{A},6^{D}-dideoxy-6^{A},6^{D}-diiodo-nonadecakis-O-benzyl-β-cyclodextrin (2). A mixture of 6^{A}, 6^{D}-diol-nonadecakis-O-benzyl-β-cyclodextrin (1, 1.40 g, 0.49 mmol, obtained as described in Pearce, A. J.; Sinaÿ, P. Angew. Chem. Int. Ed. 2000, 39, 3610-3612), iodine (749 mg, 2.95 mmol), triphenylphosphine (774 mg, 2.95 mmol) and imidazole (402 mg, 5.90 mmol) in toluene (70 mL) was vigorously stirred at 75° C. for 16 h. To reaction mixture was added an equal vol. of sat. NaHCO₃ and the mixture was stirred 5 min. Excess of iodine was removed by the addition of aqueous sat. Na₂S₂O₃. The organic layer was diluted with EtOAc (200 mL) and washed with water (80 mL), dried (MgSO₄), filtered and the organic solvent was removed in vacuo. The residue was purified by chromatography (eluent, EtOAc/Pentane 1:4→1:3), to afford 2 (1.45 g, 96%) as a white foam: [α]_{D}+34.4 (c 1.0, CHCl₃); ¹H-NMR (400 MHz, CDCl₃) δ 7.22-6.96 (m, 95H, aromatic-H), 5.24 (d, 1H, ³J_{1,2}=4.0 Hz, H-1), 5.18 (d, 1H, ³J_{1,2}=3.6 Hz, H-1), 5.10 (d, 1H, ²J=12.8 Hz, CHPh), 5.08 (d, 1H, ²J=10.8 Hz, CHPh), 5.06 (d, 1H, ³J_{1,2}=3.2 Hz, H-1), 5.04 (d, 1H, ²J=11.2 Hz, CHPh), 5.03 (d, 1H, ³J_{1,2}=3.6 Hz, H-1), 5.10 (d, 1H, ²J=10.8 Hz, CHPh), 4.91 (d, 1H, ²J=11.2 Hz, CHPh), 4.89 (d, 1H, ³J_{1,2}=3.6 Hz, H-1), 4.86 (d, 1H, ³J_{1,2}=3.6 Hz, H-1), 4.82 (d, 1H, ²J=11.2 Hz, CHPh), 4.77 (d, 1H, ²J=10.8 Hz, CHPh), 4.66-4.60 (m, 8H), 4.52-4.26 (m, 25H), 4.15 (t, 2H, ³J=8.2 Hz), 3.93-3.82 (m, 20H), 3.70-3.60 (m, 7H), 3.50-3.26 (m, 15H); ¹³C-NMR (100 MHz, CDCl₃) δ 139.5-139.2 (Cᵢₚₛₒ), 138.7-138.2 (Cᵢₚₛₒ), 128.6-127.1 (CH aromatic), 99.3 (C-1), 98.9 (C-1), 98.6 (C-1), 98.5 (C-1), 98.1 (C-1), 83.7, 81.7, 81.1, 81.0, 80.8, 80.5, 80.3, 80.1, 79.7, 79.6, 79.5, 79.0, 78.7, 78.5, 78.0, 76.2, 75.8, 75.3, 75.1, 74.9, 73.8, 73.7, 73.6, 73.2, 73.0, 72.9, 72.8, 72.7, 72.0, 71.7, 71.6, 71.2, 70.5, 69.9, 69.5, 69.4 (CH₂, CH), 9.9 (CH₂I), 9.3 (CH₂I); MALDI-TOF-MS m/z calcd for C₁₇₅H₁₈₂O₃₃I₂ 3065.0653, found 3088.0424 [M+Na]⁺.

6^{A},6^{D}-di-C-cyano-6^{A},6^{D}-dideoxy-nonadecakis-O-benzyl-β-cyclodextrin (3). Potassium cyanide (457 mg, 7.01 mmol) was added to a solution of 2 (1.02 g, 0.33 mmol) in DMF (25 mL). The reaction mixture was stirred at 80° C. for 17 h. The mixture was cooled and water (30 mL) and EtOAc (60 mL) were added. The aqueous layer was washed with EtOAc (30 mL) and the combined organic layer was washed with water (40 mL), dried (MgSO₄), filtered and the organic solvent was removed in vacuo. The residue was purified by chromatography (eluent, EtOAc/Pentane 1:3→1:2), to afford 3 (817 mg, 85%) as a white foam: [α]_{D}+35.7 (c 1.0, CHCl₃); IR (KBr) 3482, 2924, 2867, 2252 (CN), 1496, 1453, 1356, 1208, 1094, 1040 cm⁻¹; ¹H-NMR (400 MHz, CDCl₃) δ 7.29-6.89 (m, 95H, aromatic-H), 5.27 (d, 1H, ³J_{1,2}=3.6 Hz, H-1), 5.09-4.97 (m, 7H), 5.00 (d, 1H, ³J_{1,2}=3.6 Hz, H-1), 4.84 (d, 1H, ³J_{1,2}=3.0 Hz, H-1), 4.80 (d, 1H, ³J_{1,2}=3.2 Hz, H-1), 4.71-4.12 (m, 36H), 4.23-3.74 (m, 26H), 3.61 (t, 2H, ³J=11 Hz), 3.51-3.31 (m, 10H), 3.27 (dd, 1H, ³J_{2,3}=9.8 Hz, ³J_{1,2}=3.0 Hz, H-2), 2.81 (dd, 2H, ²J_{6,6′}=14.4 Hz, ³J_{5,6}=7.6 Hz, H-6_{A} or 6_{D}), 2.53 (dd, 1H, ²J_{6,6′}=17.2 Hz, ³J_{5,6}=7.2 Hz, H-6_{D} or 6_{A}), 2.47 (dd, 1H, ²J_{6,6′}=17.2 Hz, ³J_{5,6}=7.8 Hz, H-6′_{D} or 6′_{A}); ¹³C-NMR (100 MHz, CDCl₃) δ 139.5-138.1 (Cᵢₚₛₒ), 128.8-127.0 (CH aromatic), 118.1 (CN), 117.8 (CN), 99.2 (3×C-1), 98.8 (C-1), 98.5 (C-1), 98.3 (C-1), 98.0 (C-1), 82.1, 81.0, 80.8, 80.4, 80.2, 80.0, 79.6, 79.4, 79.0, 76.2, 75.8, 75.1, 74.5, 73.7, 73.5, 73.2, 73.0, 72.9, 72.8, 72.0, 71.8, 69.8, 69.5, 69.0, 67.9, 67.6 (CH₂, CH), 22.3 (CH₂CN), 21.8 (CH₂CN); MALDI-TOF-MS m/z calcd for C₁₇₇H₁₈₂O₃₃N₂ 2863.2625, found 2886.2307 [M+Na]⁺.

6^{A}, 6^{D}-di-C-cyano-6^{A}, 6^{D}-deoxy-β-cyclodextrin (4). Compound 3 (1.12 g, 0.39 mmol) was dissolved in a mixture of MeOH/EtOAc (1:1) (30 mL). Then Pd/C (112 mg) and TFA (cat) were added and the mixture was stirred over night under hydrogen atmosphere. Filtration over Celite and evaporation of the solvent gave 12 (445 mg, 99%) as a white solid: [α]_{D}+86.3 (c 1.0, H₂O); IR (KBr) 3405, 2929, 2258 (CN), 1676, 1420, 1156, 1079, 1033 cm⁻¹; ¹H-NMR (400 MHz, D₂O) δ 4.99 (d, 5H, ³J_{1,2}=3.6 Hz, H-1), 4.96 (s, 2H, H-1), 4.02 (bt, 1H, ³J=7.4 Hz, H-5), 3.78-3.68 (m, 22H), 3.61-3.47 (m, 18H), 3.44 (t, 2H, 3J=9.2 Hz), 3.37 (t, 2H, ³J=9.4 Hz), 3.08 (bd, 2H, H-6_{A} or 6_{D}), 2.83 (dd, 1H, ²J_{6,6′}=17.4 Hz, H-6_{D} or 6_{A}), 2.82 (dd, 1H, ²J_{6,6′}=16.8 Hz, H-6′_{D} or 6′_{A}); ¹³C-NMR (100 MHz, D₂O) δ 118.8 (CN), 102.2 (C-1), 102.1 (C-1), 101.9 (C-1), 84.6, 81.5, 81.4, 73.2, 72.7, 72.2, 72.0, 67.5, 60.5 (CH), 20.7 (CH₂CN); MALDI-TOF-MS m/z calcd for C₄₄H₆₈O₃₃N₂ 1152.3704, found 1175.3444 [M+Na]⁺.

6^{A}, 6^{D}-di-C-cyano-nonadecakis-O-benzyl-β-cyclodextrin (6). A mixture of KCN (649 mg, 9.96 mmol) and NH₄Cl (802 mg, 15 mmol) in water (10 mL) was added at 0° C. to a solution of 6^{A}, 6^{D}-dialdehydro-nonadecakis-O-benzyl-β-cyclodextrin (5, 200 mg, 0.07 mmol, obtained as described in Hardlei, T.; Bols, M. J. Chem. Soc. Perkin Trans 1 2002, 2880-2885) in Ether/MeOH (1:1) (10 mL). The reaction mixture was stirred overnight at room temperature. After that, the organic solvent was removed and the aqueous phase was extracted with CH₂Cl₂. The organic layer was washed, dried (MgSO₄), filtered and the organic solvent was removed in vacuo. The residue was purified by chromatography (eluent gradient, EtOAc/Pentane 1:4→1:3), to afford 6 (156 mg, 77%) as a white foam: [α]_{D}+41.8 (c 1.0, CHCl₃); IR (KBr) 3410, 3029, 2924, 2867, 2242 (CN), 1496, 1453, 1358, 1208, 1095, 1040 cm⁻¹; ¹H-NMR (400 MHz, CDCl₃), δ (ppm): 7.50-7.01 (m, 95H, aromatic-H), 5.78 (d, 1H, ³J_{1,2}=4.0 Hz, H-1), 5.75 (d, 1H, ³J_{1,2}=3.6 Hz, H-1), 5.40-5.31 (m, 4H), 5.20-5.15 (m, 2H), 5.11-4.97 (m, 6H), 4.90-4.21 (m, 44H), 4.18-3.97 (m, 14H), 3.95-3.84 (m, 5H), 3.79-3.47 (m, 15H); ¹³C-NMR (100 MHz, CDCl₃), δ (ppm): 139.9-138.0 (Cᵢₚₛₒ), 128.9-126.8 (CH aromatic), 120.3 (CN), 119.5 (CN), 100.8 (C-1), 100.2 (C-1), 99.5 (C-1), 98.3 (C-1), 98.0 (C-1), 97.5 (C-1), 97.3 (C-1), 82.3, 82.1, 81.9, 81.7, 81.4, 81.2, 81.1, 80.9, 80.6, 80.2, 79.9, 79.8, 78.9, 78.4, 76.7, 76.3, 76.2, 75.6, 75.4, 74.9, 74.6, 74.5, 74.0, 73.9, 73.7, 73.6, 73.5, 73.4, 73.2, 73.1, 73.0, 72.9, 72.7, 72.5, 72.4, 72.2, 72.1, 72.0, 71.8, 71.6, 71.1, 70.0, 69.8, 69.1, 66.1 (CH₂, CH), 60.4 (CH(OH)CN), 59.9 (CH(OH)CN); MALDI-TOF-MS m/z calcd for C₁₇₇H₁₈₂O₃₅N₂ 2895,2523, found 2918.1184 [M+Na]⁺.

(6^{A}R,6^{D}R)-6^{A},6^{D}-di-C-cyano-β-cyclodextrin (7). Compound 6 (415 mg, 0.14 mmol) was dissolved in a mixture of MeOH/EtOAc (1:1) (12 mL). Then Pd/C (42 mg) and TFA (cat.) were added and the mixture was stirred over night under hydrogen atmosphere. Filtration through Celite and evaporation of the solvent gave 7 (169 mg, 100%) as a white solid: [α]_{D}+89.4 (c 1.0, H₂O); IR (KBr) 3363, 2932, 2258 (CN), 1678, 1425, 1203, 1156, 1030 cm⁻¹; ¹H-NMR (400 MHz, D₂O) δ 5.05 (bs, 3H, H-1), 4.95 (bs, 4H, H-1), 4.00 d, 1H, ³J=8.8 Hz), 3.91-3.42 (m, 30H); ¹³C-NMR (100 MHz, D₂O) δ 119.1 (CN), 102.1 (C-1), 102.0 (C-1), 81.6, 81.5, 81.4, 81.3, 81.2, 80.0, 73.2, 73.1, 72.5, 72.3, 72.1, 72.0, 71.9 (CH), 60.7, 60.5, 59.6 (CH₂, CH(OH)CN); MALDI-TOF-MS m/z calcd for C₄₄H₆₈O₃₅N₂ 1184.3603, found 1207.3739 [M+Na]⁺.

6^{A}, 6^{D}-dicyanohydrin-hexadeckis-O-benzyl-α-cyclodextrin (9). A mixture of KCN (1.54 g, 23.57 mmol) and NH₄Cl (2.15 g, 40.17 mmol) in water (24 mL) was added at 0° C. to a solution of 6^{A}, 6^{D}-dialdehydro-hexadecakis-O-benzyl-α-cyclodextrin (8, 404 mg, 0.17 mmol, obtained as described in Hardlei, T.; Bols, M. J. Chem. Soc. Perkin Trans 1 2002, 2880-2885) in Ether/MeOH (12 mL/9 mL). The reaction mixture was stirred overnight at room temperature. After that, the organic solvent was removed and the aqueous phase was extracted with CH₂Cl₂. The organic layer was washed, dried (MgSO₄), filtered and the organic solvent was removed in vacuo. The residue was purified by chromatography (eluent gradient, EtOAc/Pentane 1:3→1:2), to afford dicyanohydrin 9 (327 mg, 80%) as a white foam: IR (KBr) 3335, 3031, 2928, 2865, 2247 (CN), 1496, 1454, 1355, 1208, 1165 cm⁻¹; ¹H-NMR (400 MHz, CDCl₃), δ (ppm): 7.42-7.14 (m, 90H, aromatic-H), 5.48 (d, 1H, ³J_{1,2}=3.6 Hz, H-1), 5.42-5.37 (m, 2H), 5.31-5.26 (m, 3H), 5.22 (d, 1H, ³J_{1,2}=3.2 Hz, H-1), 5.17 (d, 1H, ³J_{1,2}=3.2 Hz, H-1), 5.13-5.09 (m, 2H), 5.03-4.64 (m, 10H), 4.60-4.36 (m, 20H), 4.32-3.93 (m, 10H), 3.87-3.47 (m, 10H); ¹³C-NMR (100 MHz, CDCl₃), δ (ppm): 139.8-137.5 (Cᵢₚₛₒ) 129.0-126.7 (CH aromatic), 119.8 (CN), 119.7 (CN), 117.8 (CN), 101.1 (C-1), 101.0 (C-1), 100.2 (C-1), 99.8 (C-1), 99.3 (C-1), 99.1 (C-1), 98.4 (C-1), 97.8 (C-1), 82.5, 82.4, 81.9, 81.7, 81.3, 81.2, 81.1, 81.0, 80.9, 80.8, 80.5, 80.3, 79.6, 79.5, 79.3, 78.7, 76.7, 76.6, 76.2, 76.1, 75.9, 75.7, 75.3, 75.2, 73.9, 73.8, 73.7, 73.5, 73.4, 73.3, 73.2, 73.1, 73.0, 72.9, 72.8, 72.7, 72.6, 72.2, 72.0, 71.8, 71.7, 71.6, 70.4, 70.0, 69.8, 69.5, 69.4, 62.9 (CH₂, CH), 60.4 (CH(OH)CN), 60.3 (CH(OH)CN); MALDI-TOF-MS m/z calcd for C₁₅₀H₁₅₄O₃₀N₂ 2463.059, found 2486.973 [M+Na]⁺.

6^{A}, 6^{D}-dicyanohydrin-α-cyclodextrin (10). Compound 9 (327 mg, 0.13 mmol) was dissolved in a mixture of MeOH/EtOAc (1:1) (12 mL). Then Pd/C (33 mg) and TFA (cat.) were added and the mixture was stirred over night under hydrogen atmosphere. Filtration through Celite and evaporation of the solvent gave 10 (133 mg, 100%) as a white solid: ¹H-NMR (400 MHz, D₂O) δ 5.15 (bs, 1H, H-1), 5.03 (bs, 1H, H-1), 4.93 (bs, 4H, H-1), 3.95-3.39 (m, 30H).

6^{A}, 6^{D}-dialdehydro-β-cyclodextrin (11). Compound 4 (196 mg, 0.07 mmol) was dissolved in a mixture of MeOH/EtOAc (1:1) (5 mL). Then Pd/C (20 mg) and TFA (cat.) were added and the mixture was stirred over night under hydrogen atmosphere. Filtration through Celite and evaporation of the solvent gave 11 (80 mg, 100%) as a white solid: [α]_{D}+83.6 (c 1.0, H₂O); IR (KBr) 3343, 2937, 1681, 1438, 1206, 1153, 1031 cm⁻¹; ¹H-NMR (400 MHz, D₂O) δ 5.29 (bs, 2H, OH), 5.01 (bs, 2H, OH), 4.94 (bs, 7H, H-1), 3.84-3.75 (m, 31H), 3.54-3.47 (m, 20H); ¹³C-NMR (100 MHz, D₂O) δ 102.0 (C-1), 87.4, 82.3, 81.2, 73.2, 72.9, 72.2, 71.9 (CH), 60.4 (C-6); MALDI-TOF-MS m/z calcd for C₄₂H₇₀O₃₇ 1166.3596, found 1189.3035 [M+Na]⁺ (dihydrate); for C₄₂H₆₈O₃₆ 1148.3490, found 1171.2986 [M+Na]⁺ (monohydrate); for C₄₂H₆₆O₃₅ 1130.3385, found 1153.2978 [M+Na]⁺ (dialdehyde).

Hydrolysis of compound 7: To a solution of 7 (82 mg, 0.07 mmol) in water (6 mL) was added 20 mL Amberlite IR-120 (H⁺), and the mixture was stirred at 100° C. for 48 h (see the Scheme below). The resin was removed by filtration and NaBH₄ (132 mg, 3.5 mmol) was added to the filtrate. Then reaction mixture was stirred for 30 min at room temperature and Amberlite IR-120 was added until pH was acid. The resin was removed by filtration and the solvent was removed. The residue was co-evaporated with MeOH several times to remove boronic acid, to give a residue containing D-glycero-D-gluco-heptitol and D-glucitol. D-glycero-D-gluco-heptitol: ¹³C-NMR (100 MHz, D₂O) δ 72.7, 72.5, 71.6, 71.5, 69.4, 62.7, 62.3. This is in agreement with the D-glycero-D-gluco-heptitol made previously (Angyal, S. J.; Le Fur, R. Carbohydr. Res. 1984, 126, 15-26).

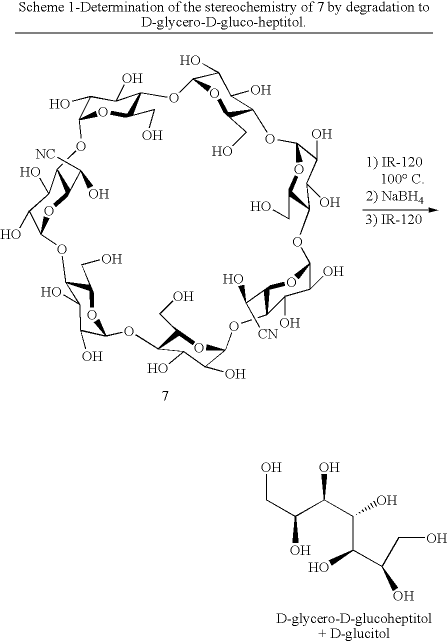

### B. Preparation of Monosubstituted Cyclodextrins

The β-cyclodextrin monoaldehyde 8 (obtained as disclosed by Rousseau, C.; Ortega-Caballero, F. Nordstrøm, L. U.; Christensen, B.; Petersen, T. E. Bols, M. Chem. Eur. J. 2005, 2734-9) was reacted with KCN giving cyanohydrin 9 in 86% yield (Scheme 2). This was followed by hydrogenolysis of the benzyl protection groups of 9 giving 3 in 73% yield. The cyanohydrin synthesis appears essentially stereoselective and a single diastereomer is in any case obtained after purification.

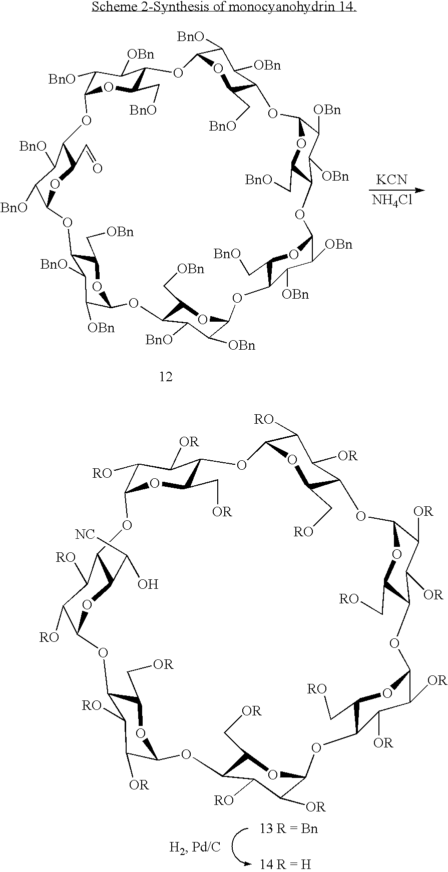

6^{A}-C-cyano-2^{A-G},3^{A-G},6^{B-G}-eiocosakis-O-benzyl-β-cyclodextrin (13). A mixture of potassium cyanide (4.08 g, 63 mmol) and ammonium chloride (5.05 g, 94 mmol) in water (63 mL) was added to a solution of 12 (1.22 g, 0.42 mmol) in Et₂O/MeOH (1:1) (63 mL). The reaction mixture was stirred overnight at room temperature. The organic solvent was removed in vacuum and the water phase was extracted with CH₂Cl₂. The organic layer was washed with water, dried (MgSO₄), filtered and concentrated in vacuo. The residue was purified by chromatography (eluent gradient, EtOAc/Pentane 1:4→1:3), to afford 13 (1.06 g, 86%) as an oil: [α]_{D}+32.6 (c 1.0, CDCl₃); IR (film) 3364, 3030, 2925, 2868, 2230 (CN), 1496, 1453, 1356, 1208, 1094, 1040, 1028 cm⁻¹; ¹H-NMR (400 MHz, CDCl₃), δ (ppm): 7.32-6.86 (m, 100H, aromatic-H), 5.59 (d, 1H, ³J_{1,2}=3.2 Hz, H-1), 5.30-5.16 (m, 3H), 5.15-4.97 (m, 6H), 4.96-4.92 (t, 2H, J=3.2 Hz), 4.91-4.88 (d, 1H, ³J_{1,2}=3.2 Hz, H-1), 4.85-4.82 (d, 1H, ³J_{1,2}=4.0 Hz, H-1), 4.76-4.24 (m, 36H), 4.08-3.77 (m, 22H), 3.76-3.38 (m, 15H), 3.40 (dd, 1H, ³J_{1,2}=3.2 Hz, ³J_{2,3}=9.6 Hz, H-2); ¹³C-NMR (100 MHz, CDCl₃), δ (ppm): 139.7-138.0 (Cᵢₚₛₒ), 129.3-125.5 (CH aromatic), 118.8 (CN), 100.0 (C1), 99.1 (C1), 98.6 (C1), 98.2 (C1), 98.0 (C1), 97.5 (C1), 81.8, 81.5, 81.4, 81.3, 81.2, 80.4, 80.0, 79.9, 79.8, 79.3, 79.2, 79.1, 78.9, 78.7, 77.8, 76.6, 76.2, 76.0, 75.6, 75.5, 74.8, 74.6, 73.9, 73.7, 73.6, 73.5, 73.5, 73.3, 73.1, 73.0, 72.9, 72.8, 72.7, 72.4, 72.3, 72.1, 72.0, 71.8, 71.6, 71.4, 71.3, 70.0, 69.9, 69.5, 69.4, 69.3, 69.2; MALDI-TOF-MS m/z calcd for C₁₈₃H₁₈₉O₃₅ NNa 2983.2937, found 2983.4310.

6-C-cyano-β-cyclodextrin (14). Compound 13 (1.06 g, 0.36 mmol) was dissolved in 2-methoxyethanol (25 mL). Then Pd/C (107 mg) and TFA (cat.) were added and the mixture was stirred at room temperature under hydrogen atmosphere until completion. Filtration through Celite and evaporation of the solvent gave 14 (303 mg, 73%) as a white solid: [α]_{D}+49.5 (c 0.1, H₂O); IR (film) 3364, 2938, 2079, 1684, 1203, 1141, 1054, 1033 cm⁻¹; ¹H-NMR (400 MHz, D₂O), δ (ppm): 5.07-5.03 (m, 1H, H-1), 5.0-4.92 (m, 6H, H-1), 4.00-3.36 (m, 41H); MALDI-TOF-MS m/z calcd for C₄₃H₆₉O₃₅NNa 1182.3548, found 1183.0570.

### C. Preparation of Monosubstituted Cyclodextrins Carrying a Further Substituent

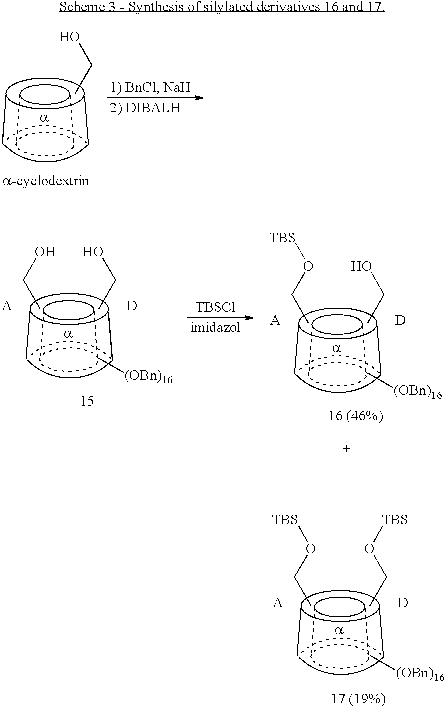

Monosilylation of the diol 15 with TBSCl/imidazol was however not very selective leading to a mixture of the mono and disilylated products 16 and 17, which could be separated by column chromatography obtain them in 46% and 19% yield, respectively.

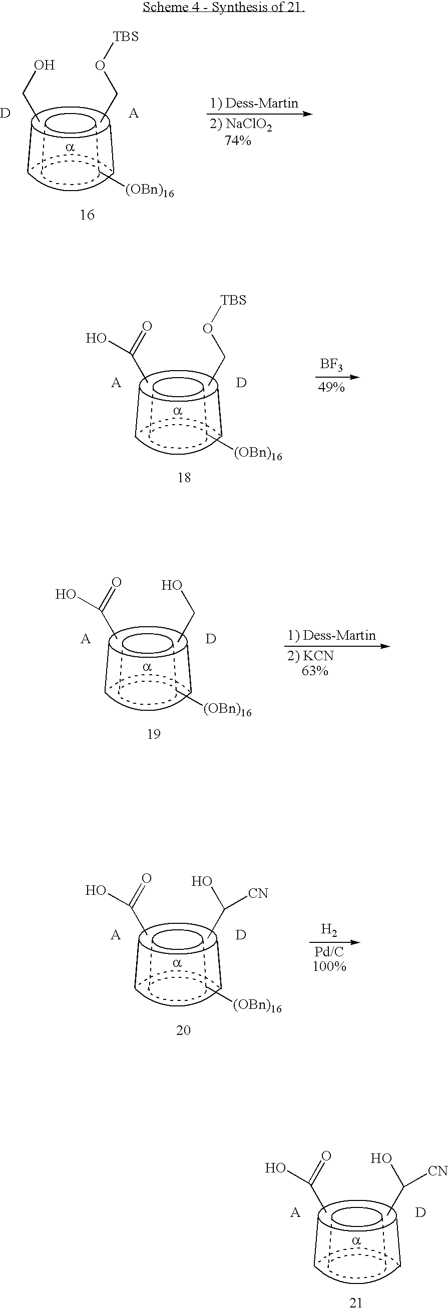

Oxidation of 16 to the aldehyde with Dess-Martin's periodinane followed by subsequent oxidation to the carboxylic acid with NaClO₂ gave the acid 18 in 74% yield (Scheme 2). Removal of the TBS group with BF₃ afforded 19 in 49% yield. Now oxidation of the remaining primary alcohol of 19 with Dess-Martin's periodinane and reaction of the resulting aldehyde with KCN gave us 20 in 63% yield. Finally, hydrogenolysis of the benzyl groups gave target 21 in quantitative yield.

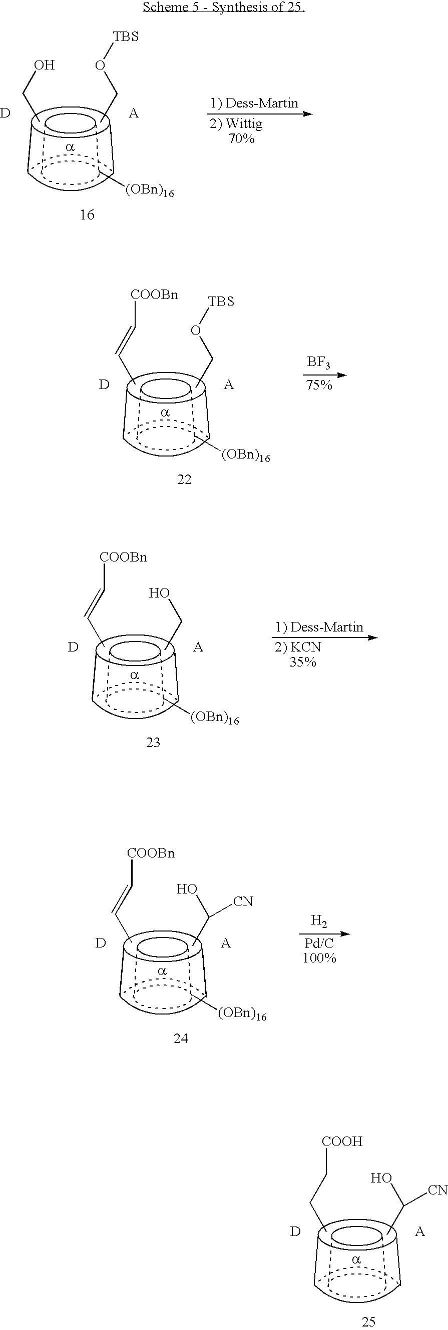

A related procedure was used to obtain 25. Oxidation of 16 to the aldehyde was followed by Wittig reaction with benzyl 2-triphenylphosphonium acetate, which led to Wittig adduct 22 in 70% yield (scheme 3). Deprotection of the silyl group with BF₃ gave the structure 23. As 23 was subjected to oxidation and reaction with KCN, the aldehyde was converted to the cyanohydrin 24 albeit in a rather low yield of 35%. Hydrogenolysis eventually gave saturation of the double bond and removal of all protection groups affording a quantitative yield of target 25.

6^{A}-tert-butyldimethylsilyl-2^{A-F},3^{A-F},6^{B},6^{C},6^{E},6^{F}-hexadecakis-O-benzyl-α-cyclodextrin (16) and 6^{A},6^{D}-di-O-tert-butyldimethylsilyl-2^{A-F},3^{A-F},6^{B},6^{C},6^{E},6^{F}-hexadecakis-O-benzyl-α-cyclodextrin (17). A solution of 15 (2.00 g, 0.83 mmol), imidazole (113 mg, 1.66 mmol) and TBDMSCl (150 mg, 0.99 mmol) in anhydrous DMF (20 mL) were stirred at room temperature under N₂ atmosphere for 48 h. The reaction mixture was diluted with EtOAc and the organic phase was washed several times with water, dried (MgSO₄), filtered and the organic solvent was removed in vacuo. The residue was purified by chromatography (eluent gradient, EtOAc/Pentane 1:4→2:7), to afford first 17 (19%) and then 16 (955 mg, 46%) as white foams. 16: [α]_{D}+32.2 (c 1.0, CH₃Cl); IR (KBr) 3482, 3063, 3029, 2926, 2857, 1951, 1733, 1605, 1496, 1453, 1359, 1094, 1027 cm⁻¹; ¹H-NMR (400 MHz, CDCl₃) δ 7.33-7.08 (m, 80H, aromatic-H), 5.57 (d, 1H, ³J_{1,2}=3.2 Hz, H-1), 5.47 (d, 1H, ³J_{1,2}=3.2 Hz, H-1), 5.36 (d, 1H, ²J=10.8 Hz, CHPh), 5.32 (d, 1H, ²J=10.8 Hz, CHPh), 5.21 (t, 2H, ²J=10.8 Hz, CH₂Ph), 5.04 (d, 1H, ³J_{1,2}=3.2 Hz, H-1), 4.95-4.80 (m, 13H), 4.70 (t, 2H, ²J=12.4 Hz), 4.59-4.34 (m, 21H), 4.25-4.08 (m, 15H), 4.00-3.81 (m, 14H), 3.73-3.60 (m, 7H), 3.56 (dd, 1H, ³J_{1,2}=3.2 Hz, ³J_{2,3}=9.6 Hz, H-2), 3.50-3.43 (m, 3H), 3.41 (dd, 1H, ³J_{1,2}=3.0 Hz, ³J_{2,3}=9.8 Hz, H-2), 3.36 (dd, 1H, ³J_{1,2}=3.0 Hz, ³J_{2,3}=9.6 Hz, H-2), 2.56 (bs, 1H, OH), 0.87 (s, 9H, SiC(CH₃)₃), 0.00 (s, 6H, SiCH₃); ¹³C-NMR (100 MHz, CDCl₃) δ 139.4-137.9 (Cᵢₚₛₒ), 128.3-126.8 (CH aromatic), 98.7 (C-1), 98.1 (C-1), 98.0 (C-1), 97.9 (C-1), 97.8 (C-1), 97.7 (C-1), 81.4, 81.3, 81.0, 80.8, 80.5, 80.0, 79.8, 79.6, 79.4, 79.2, 79.1, 79.0, 78.6, 78.2, 76.1, 76.0, 75.9, 75.8, 75.7, 75.6, 74.8, 74.6, 73.3, 73.1, 73.0, 72.9, 72.6, 72.4, 72.2, 71.9, 71.8, 71.6, 71.4, 71.3, 69.3, 69.2, 68.8 (CH₂, CH), 62.5 (C-6), 61.4 (C-6), 60.3 (C-6), 26.0 (SiC(CH₃)₃), 18.3 (SiC), −4.7 (SiCH₃), −4.9 (SiCH₃); MALDI-TOF-MS m/z calcd for C₁₅₄H₁₇₀O₃₀SiNa 2550.144, found 2550.526 [M]⁺.

17: ¹H-NMR (400 MHz, CDCl₃) δ 7.30-7.12 (m, 80H, aromatic-H), 5.31-5.20 (m, 8H), 5.14-5.11 (m, 4H), 4.91-4.86 (m, 6H), 4.62-4.41 (m, 19H), 4.33 (t, 4H, ²J=12.4 Hz), 4.23-3.94 (m, 21H), 3.80 (d, 1H, ³J=8.4 Hz), 3.66-3.50 (m, 10H), 3.42 (dd, 2H, ³J_{1,2}=3.4 Hz, ³J_{2,3}=9.4 Hz, H-2), 0.91 (s, 18H, SiC(CH₃)₃), 0.01 (s, 6H, SiCH₃), 0.00 (s, 6H, SiCH₃); ¹³C-NMR (100 MHz, CDCl₃) δ 139.3-138.1 (Cᵢₚₛₒ), 128.3-126.8 (CH aromatic), 98.3 (C-1), 98.1 (C-1), 98.0 (C-1), 81.3, 81.1, 80.7, 79.2, 79.1, 79.0, 78.1, 78.0, 75.8, 75.4, 75.3, 73.5, 73.4, 73.0, 72.8, 72.6, 72.4, 71.6, 71.4, 69.1, 69.0 (CH₂, CH), 62.3 (C-6), 26.0 (SIC(CH₃)₃), 18.2 (SiC), −4.9 (SiCH₃), −5.2 (SiCH₃).

6^{D}-O-tert-butyldimethylsilyl-2^{A-F},3^{A-F},6^{B},6^{C},6^{E},6^{F}-hexadecakis-O-benzyl-α-cyclodextrin-6^{A}-carboxylic acid (18). To a solution of 16 (336 mg, 0.13 mmol) in CH₂Cl₂ (13 mL) was added Dess-Martin periodinane reagent (141 mg, 0.33 mmol) and the reaction mixture was stirred 2 h and then quenched by addition of Et₂O (13 mL) and saturated aqueous NaHCO₃ containing 3.0 g of Na₂S₂O₃ (13 mL). After being stirring for an additional hour the solution was diluted with Et₂O (50 mL) and washed successively with saturated aqueous NaHCO₃ (30 mL) and water (30 mL). The organic phase was dried (MgSO₄), filtered and the organic solvent was removed in vacuo. The residue was dissolved in a mixture of ^{t}BuOH (9.5 mL), THF (4 mL) and 2-methyl-2-buten (4 mL) and NaClO₂ (240 mg, 2.66 mmol), and NaH₂PO₄ (266 mg) in water (4 mL) were added. The reaction mixture was stirred overnight and then quenched with 1M aqueous HCl (10 mL) and extracted with EtOAc (3×50 mL). The organic phase was dried (MgSO₄), filtered and the organic solvent was removed in vacuo. The residue was purified by chromatography (eluent, EtOAc/Pentane 2:7→2:5 and 2:5, containing 1% HCOOH), to afford 18 (251 mg, 74%) as white foam: [α]_{D}+28.6 (c 1.0, CH₃Cl); IR (KBr) 3442, 3063, 3030, 2927, 1724 (COOH), 1497, 1453, 1360, 1094, 1027 cm⁻¹; ¹H-NMR (400 MHz, CDCl₃) δ 7.38-7.10 (m, 80H, aromatic-H), 5.79 (d, 1H, ³J=3.6 Hz, H-1), 5.74 (d, 1H, ³J=3.6 Hz, H-1), 5.55 (d, 1H, ²J=10.4 Hz, CHPh), 5.51 (d, 1H, ²J=10.4 Hz, CHPh), 5.22 (d, 1H, ²J=12.0 Hz, CHPh), 5.18 (d, 1H, ²J=11.2 Hz, CHPh), 5.02-4.74 (m, 16H), 4.71-3.93 (m, 50H), 3.84-3.31 (m, 19H), 3.63 (dd, 1H, ³J_{1,2}=4.0 Hz, ³J_{2,3}=9.6 Hz, H-2), 0.88 (s, 9H, SiC(CH₃)₃), 0.00 (s, 6H, SiCH₃); ¹³C-NMR (100 MHz, CDCl₃) δ 170.9 (CO), 139.3-136.4 (Cᵢₚₛₒ), 128.7-126.3 (CH aromatic), 99.3 (C-1), 98.4 (C-1), 97.9 (C-1), 97.5 (C-1), 97.1 (C-1), 96.3 (C-1), 82.8, 81.3, 81.2, 80.8, 80.6, 80.5, 80.0, 79.9, 79.8, 79.2, 79.1, 78.9, 78.1, 77.9, 76.4, 76.2, 76.1, 75.8, 75.4, 74.1, 73.7, 73.5, 73.3, 73.2, 72.9, 72.7, 72.6, 72.2, 71.8, 71.6, 71.5, 71.2, 70.8, 70.3, 70.1, 69.5, 68.9, 68.8, 62.4 (CH₂, CH), 26.0 (SiC(CH₃)₃), 18.3 (SiC), −4.5 (SiCH₃), −4.6 (SiCH₃); MALDI-TOF-MS m/z calcd for C₁₅₄H₁₆₈O₃₁SiNa 2564.1237, found 2565.203 [M]⁺.

2^{A-F},3^{A-F},6^{B},6^{C},6^{E},6^{F}-hexadecakis-O-benzyl-α-cyclodextrin-6^{A}-carboxylic acid (19). To a solution of 18 (463 mg, 0.18 mmol) in CH₂Cl₂ (5.5 mL) was added BF₃.Et₂O (0.2 mL). The reaction mixture was stirred for 1 h and half at room temperature, diluted with CH₂Cl₂ and poured in ice-water. The mixture was basicifed with aqueous NaOH 1M and the phases was separated. The organic layer was acidified with aqueous HCl 1M, dried (MgSO₄), filtered and the organic solvent was removed in vacuo. The residue was purified by chromatography (eluent, EtOAc/Pentane 1:4, containing 1% HCOOH), to afford 19 (218 mg, 49%) as white foam: [α]_{D}+35.0 (c 0.5, CH₃Cl); IR (KBr) 3442 (OH), 3031, 2924, 1746 (COOH), 1496, 1454, 1354, 1095, 1045 cm⁻¹; ¹H-NMR (400 MHz, CDCl₃) δ 7.26-6.97 (m, 80H, aromatic-H), 5.66 (d, 1H, ³J=4.0 Hz, H-1), 5.64 (d, 1H, ³J=4.0 Hz, H-1), 5.42 (d, 1H, ²J=10.0 Hz, CHPh), 5.36 (d, 1H, ²J=10.4 Hz, CHPh), 5.07 (d, 2H, ²J=10.4 Hz, CH₂Ph), 4.85-4.76 (m, 5H), 4.73-3.89 (m, 60H), 3.79 (t, 3H, ³J=8.6 Hz), 3.74-3.46 (m, 14H), 3.42 (dd, 2H, ³J_{1,2}=3.2 Hz, ³J_{2,3}=9.6 Hz, H-2), 3.37 (d, 2H, ³J_{1,2}=3.2 Hz, ³J_{2,3}=9.6 Hz, H-2), 3.22-3.16 (m, 2H), 3.11 (t, 1H, ³J=9.0 Hz, OH); ¹³C-NMR (100 MHz, CDCl₃) δ 171.1 (CO), 139.5-136.2 (Cᵢₚₛₒ), 128.8-126.3 (CH aromatic), 99.3 (C-1), 99.0 (C-1), 98.1 (C-1), 97.9 (C-1), 97.8 (C-1), 96.5 (C-1), 83.4, 81.9, 81.6, 81.5, 81.3, 81.2, 80.9, 80.6, 80.5, 79.9, 79.5, 79.4, 79.2, 78.0, 76.6, 76.4, 76.2, 75.4, 74.1, 74.0, 73.8, 73.6, 73.5, 73.4, 73.3, 73.2, 72.8, 72.5, 72.2, 71.9, 71.8, 71.4, 71.0, 70.8, 70.2, 70.0, 69.3, 68.9, 68.1, 61.0 (CH₂, CH); MALDI-TOF-MS m/z calcd for C₁₄₈H₁₅₄O₃₁Na 2450.0372, found 2450.447 [M]⁺.

6^{D}-C-Cyano-2^{A-F},3^{A-F},6^{B},6^{C},6^{E},6^{F}-hexadecakis-O-benzyl-α-cyclodextrin-6^{A}-carboxylic acid (20). To a solution of 19 (217 mg, 0.09 mmol) in CH₂Cl₂ (9 mL) was added Dess-Martin periodinane reagent (95 mg, 0.22 mmol) and the reaction mixture was stirred 2 h and then quenched by addition of Et₂O (9 mL) and saturated aqueous NaHCO₃ containing 0.36 g of Na₂S₂O₃ (9 mL). After being stirring for an additional hour the solution was diluted with Et₂O (30 mL) and washed successively with saturated aqueous NaHCO₃ (25 mL) and water (25 mL). The organic phase was dried (MgSO₄), filtered and the organic solvent was removed in vacuo. To a solution of the residue in Et₂O (3 mL) and MeOH (5 mL) were added KCN (164 mg, 2.52 mmol), NH₄Cl (225 mg, 4.20 mmol) in water (10 mL). The reaction mixture was stirred for 20 h at room temperature. After that, the organic solvent was removed and the aqueous phase was extracted with CH₂Cl₂. The organic layer was washed with water, dried (MgSO₄), filtered and the organic solvent was removed in vacuo. The residue was purified by chromatography (eluent gradient, EtOAc/Pentane 1:4→1:4, containing 1% HCOOH), to afford 20 (130 mg, 63%) as a white foam: [α]_{D}+38.6 (c 1.0, CH₃Cl); IR (KBr) 3031, 2927, 2247 (CN), 1741 (COOH), 1497, 1454, 1355, 1096, 1045 cm⁻¹; ¹H-NMR (400 MHz, CDCl₃) δ 7.25-6.96 (m, 80H, aromatic-H), 5.71 (d, 1H, ³J=3.2 Hz, H-1), 5.62 (d, 1H, ³J=4.0 Hz, H-1), 5.39 (d, 1H, ²J=10.4 Hz, CHPh), 5.34 (d, 1H, ³J=3.6 Hz, H-1), 5.32-5.21 (m, 7H), 5.13 (d, 1H, ²J=15.6 Hz, CHPh), 5.05 (m, 2H), 4.95-4.84 (m, 3H), 4.77-4.48 (m, 40H), 4.46-4.13 (m, 42H), 4.12-3.70 (m, 54H), 3.68-3.13 (m, 35H); ¹³C-NMR (100 MHz, CDCl₃) δ 170.9 (CO), 169.9 (CO), 139.6-136.2 (Cᵢₚₛₒ), 128.9-126.2 (CH aromatic), 118.4 (CN), 117.2 (CN), 100.4 (C-1), 99.6 (C-1), 99.4 (C-1), 98.5 (C-1), 98.1 (C-1), 97.9 (C-1), 97.6 (C-1), 97.1 (C-1), 97.0 (C-1), 96.3 (C-1), 84.4, 83.6, 81.5, 81.2, 81.1, 80.9, 80.5, 80.4, 80.3, 80.1, 80.0, 79.7, 79.6, 79.5, 79.4, 79.2, 79.1, 79.0, 78.6, 78.1, 76.5, 76.4, 76.2, 76.0, 75.7, 75.6, 75.4, 74.6, 74.3, 74.2, 74.0, 73.9, 73.7, 73.6, 73.5, 73.4, 73.3, 73.2, 73.1, 73.0, 72.9, 72.8, 72.7, 72.6, 72.5, 72.4, 72.3, 72.1, 72.0, 71.7, 71.6, 71.4, 71.3, 71.2, 71.0, 70.8, 70.6, 70.4, 70.2, 70.1, 69.9, 69.5, 69.2, 68.9, 68.8, 63.0 (CH₂, CH), 60.4 (CH(OH)CN); MALDI-TOF-MS m/z calcd for C₁₄₉H₁₅₂O₃₁NNa 2474.0246, found 2474.316 [M]⁺.

6^{D}-Cyano-α-cyclodextrin-6^{A}-carboxylic acid (21). Compound 20 (130 mg, 0.05 mmol) was dissolved in a mixture of MeOH/EtOAc (1:1) (5 mL). Then Pd/C (13 mg) and TFA (cat) were added and the mixture was stirred over night under hydrogen atmosphere. Filtration over Celite and evaporation of the solvent gave 21 (54 mg, 100%) as a white solid: [α]_{D}+67.2 (c 0.5, H₂O); IR (KBr) 3423, 2942, 2246 (CN), 1679 (COOH), 1437, 1205, 1151, 1034 cm⁻¹; ¹H-NMR (400 MHz, D₂O) δ 5.14-5.07 (m, 6H, H-1), 4.02-3.63 (m, 40H), 3.32 (m, 1H); ¹³C-NMR (100 MHz, D₂O) δ 163.3 (CO), 163.0 (CO), 119.0 (CN), 101.8 (C-1), 101.7 (C-1), 101.6 (C-1), 101.5 (C-1), 101.4 (C-1), 101.3 (C-1), 100.7 (C-1), 82.0, 81.7, 81.6, 81.5, 81.4, 81.3, 81.2, 80.9, 74.8, 73.1, 73.0, 72.9, 72.7, 72.4, 72.2, 72.1, 71.9, 71.7, 71.6, 71.5, 71.3, 65.4, 60.7, 60.3, 60.0, 59.7, 68.8, 63.0 (CH, CH₂, CH(OH)CN); MALDI-TOF-MS m/z calcd for C₃₇H₅₇O₃₁NNa 1034.2812, found 1034.127 [M]⁺.

Benzyl 6^{A}-tert-butyldimethylsilyl-2^{A-F},3^{A-F},6^{B},6^{C},6^{E},6^{F}-hexadecakis-O-benzyl-α-cyclodextrin-6^{D}-propenoate (22). To a solution of BnO₂CCH₂PPhBr (496 mg, 1.01 mmol) in anhydrous THF (13 mL) was added dropwise nBuLi (0.6 mL, 0.96 mmo) and the mixture was stirred at room temperature for 1 h. the reaction mixture was cooled down to −40° C. and a solution of 16 (850 mg, 0.34 mmol) in anhydrous THF (13 mL) was added. The mixture was stirred at −40° C. for 30 min and after was allowed to reach room temperature. Ether (200 mL) was added and the mixture was washed with aqueous NH₄Cl (150 mL), water (2×100 mL) and brine (100 mL). The organic layer was dried (MgSO₄), filtered and the organic solvent was removed in vacuo. The residue was purified by chromatography (eluent gradient, EtOAc/Pentane 2:11), to afford 22 (625 mg, 70%) as a white foam: [α]_{D}+42.6 (c 1.0, CH₃Cl); IR (KBr) 3030 (C═C), 2926, 1720 (CO₂Bn), 1496, 1453, 1360, 1094, 1027 cm⁻¹; ¹H-NMR (400 MHz, CDCl₃) δ 7.50 (dd, 1H, ³J_{4,5}=4.0 Hz, ³Jₜᵣₐₙₛ=15.6 Hz, CH═), 7.44-7.16 (m, 85H, aromatic-H), 6.07 (d, 1H, ³Jₜᵣₐₙₛ=15.6 Hz, ═CHCO₂Bn), 5.42 (d, 1H, ³J_{1,2}=3.2 Hz., H-1), 5.34 (d, 1H, ²J=10.8 Hz, CHPh), 5.29-5.10 (m, 10H), 5.24 (d, 1H, ³J_{1,2}=3.2 Hz, H-1), 5.02 (d, 1H, ³J_{1,2}2=3.2 Hz, H-1), 4.98-4.87 (m, 8H), 4.65-4.40 (m, 23H), 4.28-4.11 (m, 18H), 4.08-3.95 (m, 6H), 3.87 (bd, 2H³J=8.0 Hz), 3.71-3.59 (m, 7H), 3.57-3.47 (m, 4H), 3.45-3.37 (m, 3H), 0.91 (s, 9H, SiC(CH₃)₃), 0.00 (s, 6H, SiCH₃); ¹³C-NMR (100 MHz, CDCl₃) δ 165.9 (CO), 145.8 (CH═), 139.5-137.9 (Cᵢₚₛₒ), 136.0 (Cᵢₚₛₒ—CO₂Bn), 128.7-127.1 (CH aromatic), 122.0 (═CHCO₂Bn), 99.2 (C-1), 99.0 (C-1), 98.5 (C-1), 98.2 (2×C-1), 98.1 (C-1), 84.5, 81.4, 81.3, 81.1, 80.9, 80.8, 80.1, 79.7, 79.6, 79.2, 78.7, 78.6, 78.4, 78.0, 76.0, 75.9, 75.5, 75.3, 73.7, 73.4, 73.2, 72.9, 72.8, 72.7, 72.6, 72.5, 72.1, 71.9, 71.5, 71.3, 69.5, 69.4, 68.9, 68.5, 66.3, 62.3, 60.5 (CH₂, CH); MALDI-TOF-MS m/z calcd for C₁₆₃H₁₇₆O₃₁SiNa 2680.1863, found 2680.434 [M]⁺.

Benzyl 2^{A-F},3^{A-F},6^{B},6^{C},6^{E},6^{F}-hexadecakis-O-benzyl-α-cyclodextrin-6^{D}-propenoate (23). TBAF (0.12 mL, 0.12 mmol) was added to a solution of 22 (102 mg, 0.04 mmol) in anhydrous THF (1.2 mL) at 0° C. The reaction mixture was stirred at 0° C. for 6 h. Aqueous NH₄Cl was added and the mixture was diluted with ether (50 mL) and washed successively with water (2×25 mL), brine (25 mL). The organic layer was dried (MgSO₄), filtered and the organic solvent was removed in vacuo. The residue was purified by chromatography (eluent gradient, EtOAc/Pentane 2:7), to afford 23 (73 mg, 75%) as a white foam: [α]_{D}+36.6 (c 1.0, CH₃Cl); IR (KBr) 3488 (OH), 3030 (C═C), 2924, 1722 (CO₂Bn), 1496, 1453, 1354, 1094, 1039 cm⁻¹; ¹H-NMR (400 MHz, CDCl₃) δ 7.25-6.98 (m, 86H, aromatic-H and CH═), 6.00 (d, 1H, ³Jₜᵣₐₙₛ=15.6 Hz, ═CHCO₂Bn), 5.40 (d, 1H, ³J_{1,2}=3.6 Hz., H-1), 5.29 (d, 1H, ²J=10.4 Hz, CHPh), 5.28 (d, 1H, 3J_{1,2}=4.0 Hz, H-1), 5.20-5.13 (m, 3H), 5.06 (d, 1H, ²J=10.8 Hz, CHPh), 4.92 (s, 2H), 4.90 (d, 1H, ³J_{1,2}=3.0 Hz, H-1), 4.84-4.69 (m, 11H), 4.64 (d, 1H, ³J_{1,2}=3.2 Hz, H-1), 4.58 (t, 2H, ²J=11.2 Hz), 4.46 (d, 1H, ³J_{1,2}=4.0 Hz, H-1), 4.43 (d, 1H, ³J_{1,2}=4.0 Hz, H-1), 4.39-4.20 (m, 17H), 4.15-3.95 (m, 13H), 3.89-3.66 (m, 13H), 3.61-3.57 (m, 2H), 3.50-3.33 (m, 10H), 3.31 (dd, 1H, ³J_{1,2}=3.2 Hz, ³J_{2,3}=9.6 Hz, H-2), 3.25 (dd, 1H, ³J_{1,2}=3.2 Hz, ³J_{2,3}=9.6 Hz, H-2), 2.39 (bs, 1H, OH); ¹³C-NMR (100 MHz, CDCl₃) δ 165.8 (CO), 145.8 (CH═), 139.6-137.9 (Cᵢₚₛₒ), 135.9 (Cᵢₚₛₒ—CO2Bn), 128.7-126.5 (CH aromatic), 122.2 (═CHCO₂Bn), 99.3 (C-1), 99.0 (C-1), 98.7 (C-1), 98.3 (C-1), 98.2 (C-1), 97.5 (C-1), 82.5, 81.6, 81.4, 81.3, 81.2, 80.6, 80.1, 79.9, 79.6, 79.5, 79.0, 78.7, 78.3, 76.4, 76.2, 76.1, 75.9, 75.8, 74.8, 74.5, 73.5, 73.4, 73.3, 73.2, 73.1, 73.0, 72.9, 72.5, 72.4, 72.0, 71.9, 71.8, 71.6, 69.4, 69.0, 68.9, 66.3, 61.4 (CH₂, CH); MALDI-TOF-MS m/z calcd for C₁₅₇H₁₆₂O₃₁Na 2566.0998, found 2566.760 [M]⁺.

Benzyl 6^{A}-C-Cyano-2^{A-F},3^{A-F},6^{B},6^{C},6^{E},6^{F}-hexadecakis-O-benzyl-α-cyclodextrin-6^{D}-propenoate (24). Toga solution of 23 (246 mg, 0.10 mmol) in Et₂O (3.5 mL) and MeOH (6 mL) were added KCN (189 mg, 2.91 mmol), NH₄Cl (259 mg, 4.85 mmol) in water (11.5 mL). The reaction mixture was stirred overnight at room temperature. After that, the organic solvent was removed and the aqueous phase was extracted with CH₂Cl₂. The organic layer was washed with water, dried (MgSO₄), filtered and the organic solvent was removed in vacuo. The residue was purified by chromatography (eluent gradient, EtOAc/Pentane 1:4→2:7), to afford 24 (87 mg, 35%) as a white foam: [α]_{D}+47.3 (c 0.8, CH₃Cl); IR (KBr) 3030 (C═C), 2924, 2246 (CN), 1721 (CO₂Bn), 1496, 1454, 1355, 1096, 1040 cm⁻¹; ¹H-NMR (400 MHz, CDCl₃) δ 7.38 (dd, 1H, ³J_{4,5}=4.8 Hz, ³Jₜᵣₐₙₛ=15.6 Hz, CH═), 7.33-7.09 (m, 85H, aromatic-H), 6.17 (d, 1H, ³Jₜᵣₐₙₛ=15.6 Hz, ═CHCO₂Bn), 5.30 (bd, 1H, 3j=7.6 Hz.), 5.24 (d, 1H, 2J=10.4 Hz, CH₂Ph), 5.22 (d, 1H, ³J_{1,2}=3.6 Hz, H-1), 5.18 (d, 1H, ²J=11.2 Hz, CHPh), 5.16 (d, 1H, ³J_{1,2}=3.6 Hz, H-1), 5.11 (d, 1H, ²J=10.8 Hz, CHPh), 5.08-4.98 (m, 7H), 4.91-4.76 (m, 8H), 4.67-4.57 (m, 5H), 4.51-4.39 (m, 15H), 4.35 (d, 1H, ²J=12.0 Hz), 4.30 (d, 1H, ²J=12.4 Hz), 4.18-3.77 (m, 23H), 3.71 (t, 1H, ³J_{1,2}=9.0 Hz), 3.65 (d, 1H, ²J=10.8 Hz), 3.58-3.47 (m, 8H), 3.45 (dd, 1H, 3J_{1,2}=3.2 Hz, ³J_{1,2}=9.6 Hz, H-2), 3.41 (dd, 1H, ³J_{1,2}=3.2 Hz, ³J_{2,3}=9.6 Hz, H-2); ¹³C-NMR (100 MHz, CDCl₃) δ 166.3 (CO), 146.2 (CH═), 139.5-137.8 (Cᵢₚₛₒ), 135.7 (Cᵢₚₛₒ—CO₂Bn), 128.7-126.8 (CH aromatic), 122.5 (═CHCO₂Bn), 117.5 (CN), 99.5 (C-1), 99.4 (C-1), 99.3 (C-1), 98.5 (C-1), 98.4 (C-1), 98.2 (C-1), 83.6, 80.9, 80.8, 80.4, 80.2, 80.1, 80.0, 79.6, 79.3, 79.2, 78.9, 78.7, 78.3, 77.9, 76.1, 75.5, 75.4, 74.9, 73.6, 73.5, 73.4, 73.3, 73.2, 73.1, 72.9, 72.8, 72.7, 72.0, 71.7, 71.6, 69.6, 69.5, 69.0, 68.8, 68.7, 66.4, 62.9 (CH₂, CH); MALDI-TOF-MS m/z calcd for C₁₅₈H₁₆₁O₃₁NNa 2591.0950, found 2590.501 [M]⁺.

6^{A}-C-Cyano-α-cyclodextrin-6^{D}-propanoic acid (25). Compound 24 (113 mg, 0.04 mmol) was dissolved in a mixture of MeOH/EtOAc (1:1) (5 mL). Then Pd/C (12 mg) and TFA (cat) were added and the mixture was stirred over night under hydrogen atmosphere. Filtration over Celite and evaporation of the solvent gave 25 (46 mg, 100%) as a white solid: [α]_{D}+56.8 (c 1.0, H₂O); IR (KBr) 3431, 2247 (CN), 1685 (COOH), 1437, 1207, 1144, 1050 cm⁻¹; ¹H-NMR (400 MHz, D₂O) δ 4.94 (m, 6H, H-1), 3.89-3.77 (m, 20H), 3.49 (bs, 10H), 3.24-3.17 (m, 2H), 2.42 (bs, 1H), 2.29 (bs, 1H), 1.64 (bs, 2H); ¹³C-NMR (100 MHz, D₂O) δ 163.3 (CO), 163.0 (CO), 118.0 (CN), 101.7 (C-1), 101.3 (C-1), 85.9, 81.6, 81.2, 73.3, 72.0, 71.8, 71.7, 70.6, 60.4, 39.5, 26.6 (CH, CH₂, CH(OH)CN); MALDI-TOF-MS m/z calcd for C₃₉H₆₁O₃₁NNa 1062.3125, found 1062.284 [M]⁺.

### EXAMPLE 2

### Demonstration of Catalytic Effect on Hydrolysis of Glycosides. Experimental Protocol for Determining Catalysis and Results

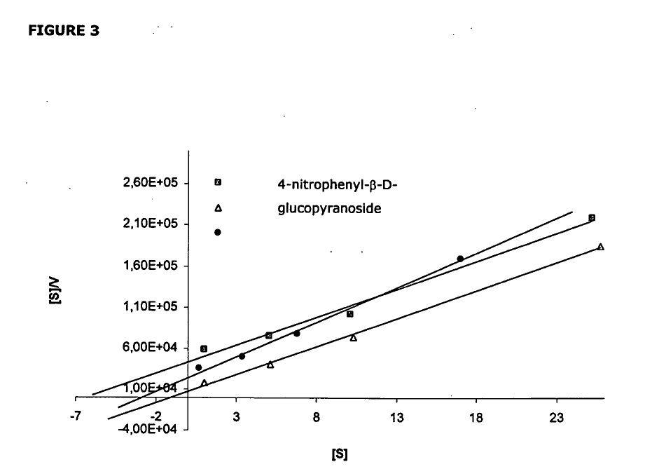

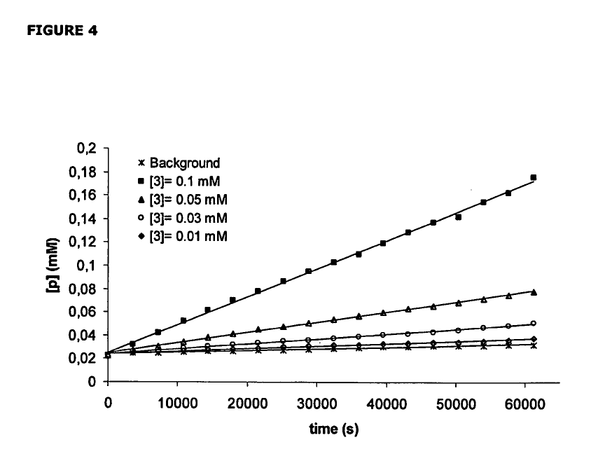

Each assay was performed on 2 mL samples prepared from 1 mL aqueous solutions of the appropriate nitrophenylglycoside at different concentrations mixed with 1 mL of 0.1 M phosphate buffer containing 7 of example 1 (0.025 mg-5 mg) or nothing as control. The reactions were followed at 59° C. using UV absorption at 400 nm and typically monitored for 18 h (see FIG. 4). Velocities were determined as the slope of the progress curve of each reaction. Uncatalysed velocities were obtained directly from the control samples. Catalyzed velocities were calculated by subtracting the uncatalysed velocity from the velocity of the appropriate cyclodextrin containing sample. The catalyzed velocities were used to construct Hanes plot ([S]/V vs. [S], see FIG. 3) from which Kₘ and Vₘₐₓ were determined. k_{cat} was calculated as Vₘₐₓ/[cyclodextrin]. k_{uncat} was determined as the slope from a plot of V_{uncat} versus [S]. The extinction coefficients 15.3 mM⁻¹cm⁻¹ (pH 7.4, 59° C.) was determined for 4-nitrophenolate and used in the calculations.

Table 1 shows the results obtained with compound 7 of example 1 on catalysis of hydrolysis of different 4-nitrophenyl glycosides. The effect is compared with experiments without the catalyst. The results clearly show that 7 is a remarkable catalyst of glycoside hydrolysis.

TABLE 1
Kinetic parameters for the 7-catalysed hydrolysis of
various glycosides in the presence of 0.42 mM 7 at pH
7.4 and 59° C. in a 50 mM phosphate buffer.

| Substrate | Kₘ (mM) | k_{cat} (×10⁵ s⁻¹) | k_{cat}/k_{uncat} |
|---|---|---|---|
| 4-nitrophenyl-β-D-glucoside | 5.4 | 3.0 | 1047 |
| 4-nitrophenyl-α-D-glucoside | 12 | 2.9 | 2147 |
| 4-nitrophenyl-α-D-mannoside | 2.8 | 1.8 | 283 |
| 4-nitrophenyl-α-D-galactoside | 1.0 | 2.3 | 486 |
| 2-nitrophenyl-β-D-galactoside | 4.2 | 6.7 | 755 |

The catalysis can be inhibited by addition of cyclopentanol confirming that the cyclodextrin cavity is involved in the process. No catalysis is observed with neither β-cyclodextrin nor mandelonitrile showing that the supramolecular positioning of binding cavity and cyanohydrin group is essential for catalysis. The kinetic experiments were carried out with 4-nitrophenyl-β-D-glucopyranoside in concentrations 1-25 mM and 7 in concentrations from 0.01-0.1 mM (FIG. 4). At these conditions (k_{cat}=3.0×10⁻⁵s⁻¹, turnover time=33000 s), it is seen that even after 2 catalytic turnovers the catalytic rate is unchanged, which confirms true catalysis.

The catalysis by 7 of the hydrolysis of other nitrophenyl glycosides was also studied (Table 1). The catalysis of the α-glucoside has essentially the same rate as the β-glucoside while the α-manno and α-galacto configured substrates are hydrolysed with a slightly slower rate. The 2-nitrophenyl-β-galactopyranoside is hydrolysed with the highest rate, but the variation in rate is nevertheless small and slightly smaller that the variation in background hydrolysis rate of the substrates.

Compound 10 is perhaps an even better catalyst. As is seen in Table 2, a k_{cat} over kuncat of up to 7000 is obtained with this catalyst.

TABLE 2
Kinetic parameters for the 10-catalysed hydrolysis of various
glycosides in the presence of 0.1-0.4 mM 10 at variant pH
and 59° C. in phosphate and other buffers.

| | | Phos- | | | |
|---|---|---|---|---|---|
| | | phate | K_{cat} | | k_{cat}/ |
| Substrate | pH | (mM) | (10⁻⁵ s⁻¹) | Kₘ | k_{uncat} |
| 4-Nitrophenyl-β-D- | 6.2 | 50 | 1.04 ± 0.03 | 10.42 ± 0.87 | 255 |
| Glucopyranoside | | | | | |
| 4-Nitrophenyl-β-D- | 6.6 | 50 | 1.47 ± 0.09 | 10.58 ± 1.69 | 862 |
| Glucopyranoside | | | | | |
| 4-Nitrophenyl-β-D- | 7.0 | 50 | 2.13 ± 0.04 | 4.43 ± 0.49 | 1001 |
| Glucopyransoside | | | | | |
| 4-Nitrophenyl-β-D- | 7.4 | 50 | 4.57 ± 0.09 | 4.76 ± 0.27 | 1654 |
| Glucopyranoside | | | | | |
| 4-Nitrophenyl-β-D- | 7.7 | 50 | 5.39 ± 0.51 | 8.94 ± 2.48 | 2651 |
| Glucopyranoside | | | | | |
| 4-Nitrophenyl-β-D- | 8.0 | 50 | 7.14 ± 0.26 | 7.75 ± 1.00 | 2755 |
| Glucopyranoside | | | | | |
| Hellicin | 7.4 | 50 | 5.20 ± 1.94 | 6.38 ± 3.72 | 93 |
| 4-Nitrophenyl-β-D- | 7.4 | 100 | 4.90 ± 0.39 | 5.56 ± 1.02 | 2316 |
| Glucopyranoside | | | | | |
| 4-Nitrophenyl-β-D- | 7.4 | 250 | 2.98 ± 0.54 | 3.23 ± 0.24 | 804 |
| Glucopyranoside | | | | | |
| 4-Nitrophenyl-β-D- | 8.0 | 500 | 20.3 ± 3.41 | 8.66 ± 4.08 | 7116 |
| Glucopyranoside | | | | | |
| 4-Nitrophenyl-β-D- | 8.0 | Borate- | 1.31 ± 0.00 | 8.33 ± 0.17 | 959 |
| Glucopyranoside | | HCl | | | |
| 4-Nitrophenyl-β-D- | 8.9 | Borate- | 0.42 ± 0.13 | — | 151 |
| Glucopyranoside | | HCl | | | |
| 4-Nitrophenyl-β-D- | 7.4 | HEPES | 0.39 ± 0.03 | 15.60 ± 0.99 | 204 |
| Glucopyranoside | | | | | |
| 4-Nitrophenyl-β-D- | 8.5 | Glycin- | 1.31 ± 1.00 | 36.03 ± 12.33 | 432 |
| Glucopyranoside | | NaOH | | | |

To get insight into the nature of the catalysis, cyclodextrin analogues 4 (dinitrile) and 11 (dialdehyde hydrate) were prepared and their catalytic ability studied. In Table 3 below is shown the results of different cyclodextrin derivatives of the invention and comparison with ordinary cyclodextrins. The results clearly show that 7 is a much more potent catalyst than ordinary cyclodextrins and the corresponding dinitrile (4) and dialdehyde hydrate (11).

TABLE 3
Kinetic parameters for the catalysis by different cyclodextrin
derivatives of the hydrolysis of 4-nitrophenyl-β-D-glucoside
at pH 7.4 and 59° C. in a 50 mM phosphate buffer.

| Catalyst | Kₘ (mM) | k_{cat} (×10⁵ s⁻¹) | k_{cat}/k_{uncat} |
|---|---|---|---|
| 7 (0.42 mM) | 5.4 | 3.0 | 1047 |
| α-cyclodextrin | — | — | — |
| β-cyclodextrin | — | — | — |
| 4 (2.2 mM) | 6.3 | 0.011 | 4 |
| 11 (0.44 mM) | 7.6 | 0.14 | 48 |

The catalytic power of these compounds towards 4-nitrophenyl-β-D-glucoside hydrolysis is shown in Table 2. The native cyclodextrins afforded no catalysis. This shows that the cyano groups are essential. Dinitrile 4 is catalytic, but with a 250 times lower catalytic power, showing that the two cyanohydrin OH groups are very important for the catalysis. Finally, the dialdehyde 11, which NMR shows is exclusively on dihydrate form in aqueous solution, is a catalyst with a catalytic efficacy of 20 times lower than 7.

Based on these results, the following can be elucidated about the catalysis. Previous work on 6-C-substituted cyclodextrins concluded, based on modeling and the highly variant polarity of the 6S and 6R isomers, that these derivatives have very restricted conformational freedom along the C5-C6 bond as both OH and alkyl substituents shun the tg conformation (see Hardlei, T.; Bols, M. J. Chem. Soc. Perkin Trans 1 2002, 2880-2885). Therefore an important feature in 7 is that the cyanohydrin 6-OH groups are fixed in the gt conformation pointing towards the binding site. These hydroxy groups appear to be essential. The role of the cyano groups must be to draw electrons away from the OH groups making them more acidic. This is supported by the observation that 11 is the second best catalyst. In this compound, the cyano groups have been replaced by OH groups, which are electron-withdrawing though less so. Thus, an increased acidity of these OH groups appears to be a crucial factor. This fits a role of the OH groups acting as general acids. We therefore propose a mechanism for the catalysis as outlined in FIG. 5. A cyanohydrin OH group donates a proton to the exocyclic oxygen facilitating cleavage.

The compound defined herein exemplified by the cyclodextrin cyanohydrin 7 are encouragingly potent catalysts and appear to mimic part of mechanistic apparatus of natural glycosidases though with an entirely different functionality, the cyanohydrin.

TABLE 1
Kinetic paramaters for the dicyanohydrin-β-cyclodextrin (7)-catalysed hydrolysis of
various glycosides at different pH and 59° C. The reactions were followed by measuring
absorption at 400 nm. * at 25° C. †at 90° C. # Followed by measuring absorption at 290 nm.
§ Followed by measuring glucose formation. @ in D₂O. Concentration of 7 was 0.42 mM.

| | | Phosphate | k_{cat} | Kₘ | k_{cat}/ |
|---|---|---|---|---|---|
| Substrate | pH | (mM) | (x10⁻⁵s⁻¹) | (mM) | k_{uncat} |
| 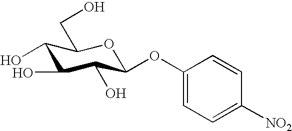 | 6.2 6.6 7.0 7.4 7.7 8.0 8.0 8.0 8.0 8.0 8.0 8.0 7.4* 8.0^{@} | 50 50 50 50 50 50 25 100 175 250 350 500 50 50 | 1.03 ± 0.21 1.83 ± 0.02 3.13 ± 0.05 5.02 ± 0.34 5.55 ± 0.26 5.87 ± 0.73 6.66 ± 0.34 6.32 ± 0.47 9.02 ± 0.45 12.3 ± 0.43 14.7 ± 0.41 14.2 ± 0.68 0.40 ± 0.03 4.04 ± 0.16 | 4.14 ± 3.64 4.97 ± 1.21 5.90 ± 0.43 4.60 ± 1.48 4.54 ± 1.04 10.32 ± 4.15 3.61 ± 0.97 3.34 ± 1.33 5.26 ± 1.23 7.75 ± 1.02 8.74 ± 0.87 6.25 ± 1.26 6.67 ± 1.17 3.10 ± 0.68 | 421 989 2577 3141 2247 3116 2487 2055 3212 5340 5759 6396 920 2888 |
| 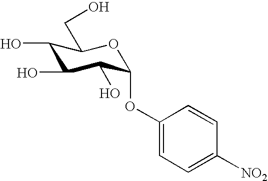 | 7.4 8.0 | 50 500 | 3.05 ± 0.41 14.2 ± 0.9 | 12.7 ± 3.7 10.5 ± 1.5 | 2234 7922 |
| 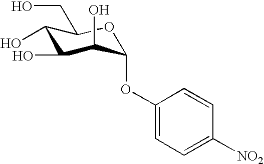 | 7.4 | 50 | 1.84 ± 0.11 | 2.65 ± 0.55 | 279 |
| 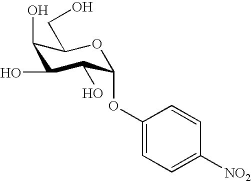 | 7.4 | 50 | 2.40 ± 0.12 | 1.46 ± 0.36 | 513 |
| 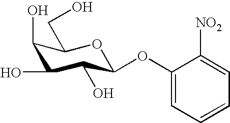 | 7.4 | 50 | 4.52 ± 0.01 | 1.62 ± 1.55 | 512 |
| 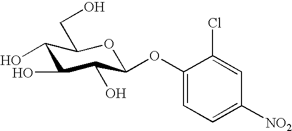 | 8.0 | 50 | 5.26 ± 0.41 | 7.17 ± 1.30 | 51 |
| 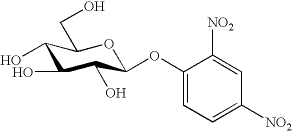 | 8.0* | 50 | 0.93 ± 0.19 | 5.44 ± 2.82 | 6 |
| 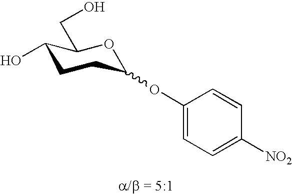 | 8.0* | 50 | No catalysis | | — |
| 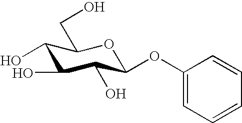 | 8.0^{†} | 50 | 3.30 ± 0.21^{#} 3.27 ± 0.15^{§} | 3.03 ± 0.88^{#} 0.63 ± 0.19^{§} | Not dtmnd 1200 |
| 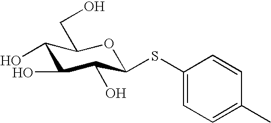 | 8.0 | 50 | 18.2 ± 0.08 | 0.06 ± 0.03 | 19 |

TABLE 2
Kinetic parameters for the dicyanohydrin-α-cyclodextrin 10-catalysed hydrolysis of
various glycosides at different pH and 59° C. The reactions were followed by measuring
absorption at 400 nm. * at 25° C. # Followed by measuring absorption at 376 nm.
Concentration of 10 was 0.49 mM.

| | | Phosphate | k_{cat} | Kₘ | k_{cat}/ |
|---|---|---|---|---|---|
| Substrate | pH | (mM) | (10⁻⁵s⁻¹) | (mM) | k_{uncat} |
| 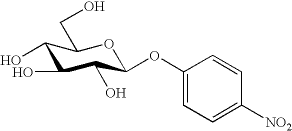 | 6.2 6.6 7.0 7.4 7.7 8.0 7.4 7.4 8.0 8.0 8.9 7.4 8.5 | 50 50 50 50 50 50 100 250 500 Borate-HCl Borate-HCl HEPES Glycin-NaOH | 0.93 ± 0.05 1.44 ± 0.07 2.16 ± 0.05 4.59 ± 0.10 5.34 ± 0.46 6.96 ± 0.29 4.82 ± 0.33 3.01 ± 0.08 20.3 ± 2.9 1.31 ± 0.00 0.64 ± 0.10 0.30 ± 0.03 0.89 ± 0.17 | 6.34 ± 1.04 9.30 ± 1.53 4.50 ± 0.47 4.69 ± 0.33 7.56 ± 2.50 6.52 ± 1.10 4.85 ± 1.05 3.34 ± 0.34 6.63 ± 3.86 8.09 ± 0.18 — 7.78 ± 1.35 12.32 ± 5.21 | 227 849 1013 1660 2629 2688 2279 812 7101 955 227 157 277 |
| 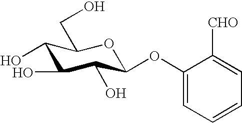 | 7.4 | 50 | 5.31 ± 1.21^{#} | 4.51 ± 2.70^{#} | 95 |
| 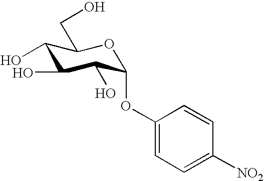 | 8.0 | 500 | 16.5 ± 2.0 | 14.8 ± 3.6 | 6138 |
| 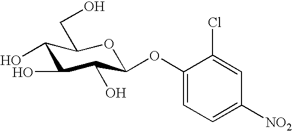 | 8.0 | 50 | 3.70 ± 0.68 | 7.12 ± 3.04 | 58 |
| 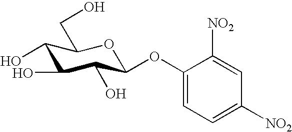 | 8.0* | 50 | 0.54 ± 0.03* | 7.77 ± 0.77* | 4 |
| 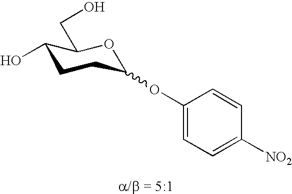 | 8.0* | 50 | No catalysis | | — |

TABLE 3
Kinetic parameters for the cyanohydrin-β-cyclodextrin (14)-catalysed hydrolysis of
various glycosides at different phosphate concentrations and 59° C. The reactions were
followed by measuring absorption at 400 nm. Concentration of 14 was 0.43 mM.

| | | Phosphate | k_{cat} | Kₘ | k_{cat}/ |
|---|---|---|---|---|---|
| Substrate | pH | (mM) | (10⁻⁵s⁻¹) | (mM) | k_{uncat} |
| 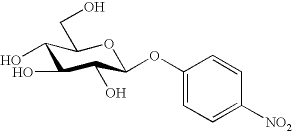 | 8.0 8.0 | 50 500 | 1.47 ± 0.12 2.88 ± 0.48 | 30.6 ± 4.9 4.69 ± 3.09 | 1067 1356 |
| 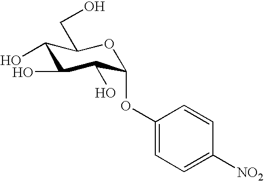 | 8.0 8.0 | 50 500 | 0.69 ± 0.12 7.56 ± 2.32 | 2.10 ± 1.46 12.7 ± 7.8 | 311 1299 |
| 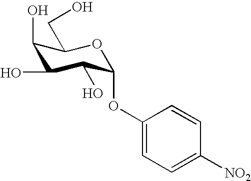 | 8.0 | 50 | 0.39 ± 0.08 | 0.00 ± 0.96 | 100 |
| 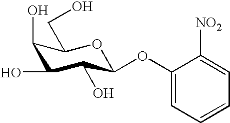 | 8.0 | 50 | 0.17 ± 0.03 | 10.0 ± 3.4 | 14 |

1. A compound having a cyclodextrin skeleton wherein a hydrogen atom at the C-6 position in at least one of the sugar moieties has been substituted with a cyano group.

2. The compound according to claim 1, which comprises 5-10 sugar moieties, in particular 6-8 sugar moieties.

3. The compound according to claim 1, having the general structure I 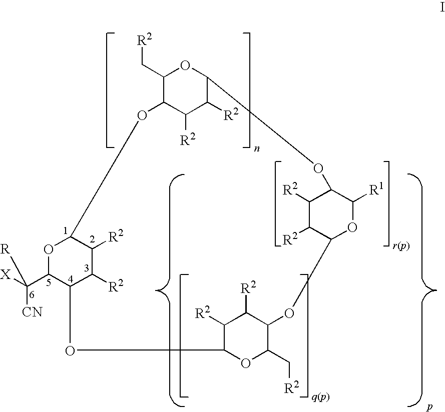
wherein
n is an integer of 0-9,
each q(p) is an integer of 0-2,
each r(p) is an integer of 1-2, and
p is an integer of 0-5,
with the proviso that the sum (1+n+Σ{q(x)+r(x)}_{x=1, . . . , p}) is 5-10;
each R independently is selected from hydrogen, cyano, hydroxy, C₁₋₈-alkyl; C₃₋₈-cycloalkyl, C₂₋₈-alkenyl, CF₃, C₁₋₈-alkylcarbonyloxy, carboxy, and mono- or di(C₁₋₈-alkyl)aminocarbonyl;
each X independently is selected from heteroatom substituents;
each R¹ independently is selected from optionally substituted C₁₋₈-alkyl, optionally substituted C₃₋₈-cycloalkyl, optionally substituted C₂₋₈-alkenyl, mono- or di-(C₁₋₄-alkyl)amino, tri(C₁₋₈-alkyl)ammonium, carboxy, carboxaldehyde, optionally substituted aryl, and a group 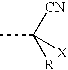
where R and X are defined as above (in particular CH(OH)CN);
each R² independently is selected from hydrogen, hydroxy, optionally substituted C₁₋₈-alkoxy, optionally substituted aryloxy, optionally substituted arylmethyloxy, optionally substituted C₁₋₈-acyloxy, tri-substituted silyloxy, O-phosphate and O-sulphate, or two R² substituents on neighbouring carbon atoms form an O,O-acetal group;
and salts thereof.

4. The compound according to claim 3, wherein the sum (1+n+Σ{q(x)+r(x)}_{x=1, . . . , p}) is 6-8.

5. The compound according to claim 3, wherein
each X independently is selected from hydroxy, amino, thio, C₁₋₈-alkylamino, and C₃₋₈-cycloalkylamino; and
each R¹ independently is a group 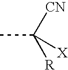
where R and X are defined as above, in particular a group CH(OH)CN.

6. The compound according to claim 3, having the general structure II 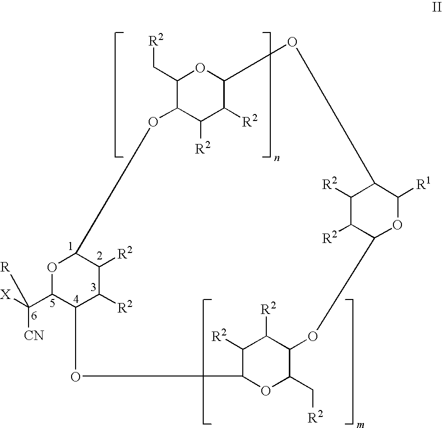
wherein n, R, X, R¹ and R² are as defined above, and
m is an integer from 0 to 4, with the proviso that the sum (2+m+n) is 5-10, in particular 6-8.

7. The compound according to claim 3, wherein
each R¹ independently is a group 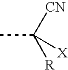
where R and X are defined as above, in particular a group CH(OH)CN.

8. The compound according to claim 3, wherein
each X independently is selected from hydroxy, thio and amino;
each R is hydrogen; and
each R¹ independently is selected from CH(OH)CN, CH(SH)CN and CH(NH₂)CN, in particular CH(OH)CN.

9. The compound according to claim 3, wherein each R² independently is selected from hydrogen, hydroxy, and C₁₋₄-alkoxy, in particular hydroxy.

10. The compound according to claim 3, wherein
each X is OH;
each R independently is selected from hydrogen, C₁₋₈-alkyl and C₃₋₈-cycloalkyl;
each R¹ independently is selected from C₁₋₈-alkyl optionally substituted with hydroxy or C₁₋₄alkoxy, carboxy, carboxaldehyde, and the group 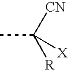
where R and X are defined as above, in particular CH(OH)CN; and
each R² independently is selected from hydroxy and C₁₋₄-methoxy.

11. The compound according to claim 6, wherein
X is OH;
R is hydrogen;
R¹ is CH(OH)CN;
R² is hydroxy; and
m is 2 and n is 2 or 3.

12. The compound according to claim 1, which has the structure III

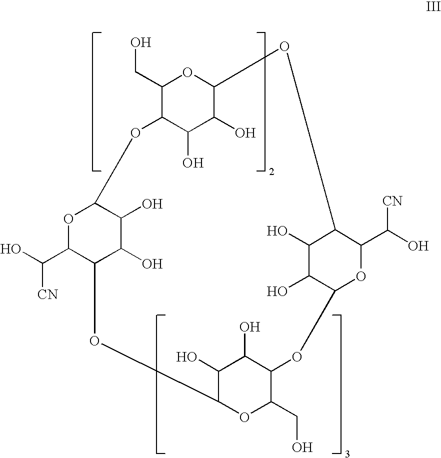

13. The compound according to claim 1, which has the structure IV

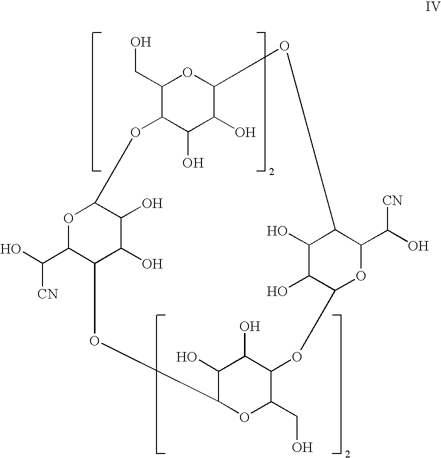

14. The use of a compound according to claim 1 as a catalyst.

15. A method of hydrolysing an aryl glycoside, wherein said hydrolysis is carried out in the presence of a compound according to claim 1.

16. A solid phase material having immobilized thereto a compound according to claim 1.

17. A method of reducing or eliminating the content of aryl glycosides in a composition, said method comprising the steps of contacting the composition with a compound according to claim 1, or a solid phase material according to claim 22, under conditions suitable for effecting hydrolysis of said aryl glycosides.

18. The method according to claim 17, wherein said composition is a foodstuff.

19. A pharmaceutical composition comprising a compound according to claim 1, and a pharmaceutically acceptable carrier, excipient or diluent therefor.

20. A compound according to claim 1 for use as a medicament.

21. The use of a compound according to claim 1 for the preparation of a medicament for the treatment of poisoning.

22. The use of a compound according to claim 1 for the preparation of a medicament for the treatment of drug addiction.

23. A method of treating a mammal suffering from poisoning, said method comprising the step of administering a compound according to claim 1 to said mammal.

24. A method of treating a mammal suffering from drug addiction, said method comprising the step of administering a compound according to claim 1 to said mammal.
